(19) United States
(12) Patent Application Publication
(10) Pub. No.: US 2015/0064695 A1
(43) Pub. Date: Mar. 5, 2015

(54) METHODS FOR SCREENING AND DIAGNOSING GENETIC CONDITIONS

(71) Applicants: Andrew S Katz, La Jolla, CA (US); Andrew E Senyei, La Jolla, CA (US)

(72) Inventors: Andrew S Katz, La Jolla, CA (US); Andrew E Senyei, La Jolla, CA (US)

(73) Assignee: Celula Inc., San Diego, CA (US)

(21) Appl. No.: 13/993,306

(22) PCT Filed: Dec. 16, 2011
(86) PCT No.: PCT/US11/65642

§ 371 Date: Nov. 13, 2014

Related U.S. Application Data
(60) Provisional application No. 61/424,597, filed on Dec. 17, 2010.

(51) Int. Cl. C12Q 1/68

(52) U.S. Cl. CPC C12Q 1/6883; C12Q 2600/156
USPC 435/6.11

(57) ABSTRACT

The present invention relates to methods and systems useful for screening and/or diagnosing genetic conditions in a fetus.

### RELATED APPLICATIONS

The present application claims priority to U.S. Provisional Patent Application Ser. No. 61/424,597, filed on Dec. 17, 2010, which is hereby incorporated by reference herein in its entirety.

### BACKGROUND OF THE INVENTION

1. Field of the Invention

Embodiments of the present invention relate to methods and systems useful for screening and/or diagnosing genetic conditions in a fetus.

2. Description of the Related Art

Prenatal screening and diagnostic testing involves testing the fetus before birth (prenatally) to predict or determine whether the fetus has certain genetic variations, including certain genetic conditions. Current screening tests, such as ultrasonography and certain blood tests, are often part of routine prenatal care. The goal of a screening test is to identify a fetus with a sufficiently high risk of a genetic condition (e.g., Down syndrome) to justify further invasive tests which are diagnostic of the genetic condition. Invasive diagnostic tests (e.g., chorionic villus sampling or amniocentesis) involve sampling procedures that carry a certain risk to the mother and/or fetus including the induction of miscarriage and fetal limb defects. There is, therefore, a need for screening tests that maximize the chance of identifying or detecting those pregnancies at highest risk of having a genetic condition, so as to justify further invasive diagnostic tests.

The effectiveness of a prenatal screening test depends on its ability to discriminate between pregnancies with genetic conditions and unaffected pregnancies. The performance of a screening test is usually specified in terms of its sensitivity (i.e., detection rate) and specificity (i.e., false-positive rate). Various combinations of biochemical and ultrasound markers are currently used together at different stages of pregnancy.

For example, the “combined test” carried out in the first trimester using nuchal translucency and free β-human chorionic gonadotropin (Fβ-hCG) and pregnancy-associated plasma protein A (PAPP-A) as screening markers can achieve a 91% detection rate with a 5% false-positive rate. (Perni et al. (2006) Am J Obstet Gynecol. 194:127-130). The “triple test” carried out in the second trimester uses alpha-fetoprotein (AFP), unconjugated estriol (uE) and hCG as screening markers. The “triple test” can achieve a 91% detection rate with a false positive rate of 5.6%. (Palka et al. (1998) Minerva Ginecol. 50:411-5). The high false-positive rates observed in current testing protocols results in a large number of women with unaffected pregnancies being informed that their fetus is positive for a genetic condition when, in fact, it is not. For these unaffected women the screen-positive result, in addition to causing considerable anxiety, might lead to a diagnostic procedure such as amniocentesis or chorionic villus sampling which can have a risk of miscarriage of about 1 in 100. Other drawbacks with current prenatal screening tests include: multiple office visits, numerous blood draws, and time to diagnosis (weeks to months). Accordingly, there is a need in the art for improved prenatal screening methods.

### SUMMARY OF THE INVENTION

Embodiments of the present invention provide methods and systems for screening and/or detecting a genetic condition in a fetus using various reflex protocols of biochemical marker measurement steps and fetal genetic variation detection steps in which subsequent biochemical marker measurement steps and/or fetal genetic variation detection steps are selectively performed based on the results of previous biochemical marker measurements or fetal genetic variation detection and without the need for human decision making. Reflex protocols (i.e., protocols which specify selection of subsequent tests based on results of previous tests, without the need for subjective human decision-making in selecting tests) have been employed in the assessment of medical conditions such as thyroid disease (See, e.g., Klee et al. (1987) J Clin Endocrinol. 64:641-671) and for identifying individuals at high risk of cancer (See, e.g., Fraser et al. (2007) Gut 56:1415-1418). However, reflex protocols for the screening or detection of fetal genetic conditions have not yet been employed. Thus, reflex protocols and the use of such protocols for the screening or diagnosis of fetal genetic conditions are needed in the art to improve the fetal diagnostic process through their efficiency, effectiveness and reduction of costs of further unwarranted testing.

One embodiment of the invention is a method providing improved accuracy of information for a patient regarding her fetus, comprising: selecting a plurality of different prenatal tests comprising at least a first prenatal test and a second prenatal test for a prenatal screen of a patient's fetus; setting a minimum detection rate and a maximum false positive rate for the prenatal screen comprising the plurality of prenatal tests; adjusting the threshold required for a positive test result for the first prenatal test such that the false positive rate for the first prenatal test exceeds the maximum false positive rate for the prenatal screen; adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen; obtaining a biological sample from a patient identified as the source of a test result that meets the adjusted threshold required for a positive test result for the first prenatal test; subjecting the biological sample from the patient to the second prenatal test to determine a level of a biological marker in the biological sample, where the level of the biological marker constitutes a second prenatal test result; determining whether the second prenatal test result meets the adjusted threshold required for a positive test result for the second prenatal test; sequentially subjecting a biological sample from the patient to testing in any remaining plurality of prenatal tests if the test result for the biological sample meets the adjusted threshold required for a positive test result for the second and any subsequent prenatal test; and identifying the patient as having a positive prenatal screen result if the test results for each of the plurality of prenatal tests to which a biological sample is subjected meets the adjusted threshold required for a positive test result for each of the plurality of prenatal tests, where the false positive rate for the prenatal screen at a given detection rate is less than the false positive rate for any of the plurality of prenatal tests alone, thereby improving the accuracy of information from the prenatal screen of the patient's fetus.

Another embodiment of the invention is a method of increasing the cost effectiveness of a prenatal screen, comprising: selecting a plurality of different prenatal tests comprising at least a first prenatal test and a second prenatal test for a prenatal screen; setting a minimum detection rate and a maximum false positive rate for the prenatal screen comprising the plurality of prenatal tests; adjusting the threshold required for a positive test result for the first prenatal test such that the false positive rate for the first prenatal test exceeds the maximum false positive rate for the prenatal screen; adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen; generating a first prenatal test result for biological samples from a plurality of patients; identifying a subset of the biological samples that meets the adjusted threshold required for a positive test result for the first prenatal test and a subset of the biological samples that does not meet the adjusted threshold required for a positive test result for the first prenatal test; generating a second prenatal test result for the subset of samples that meets the adjusted threshold required for a positive test result for the first prenatal test; determining whether the second prenatal test result meets the adjusted threshold required for a positive test result for the second prenatal test; sequentially subjecting samples from the subset of samples that meets the adjusted threshold required for a positive test result for the second prenatal test to testing in any remaining plurality of prenatal tests if the biological samples meet the adjusted threshold required for a positive test result for any subsequent prenatal test; and identifying patients as having a positive prenatal screen result if the test result for the final prenatal test to which their biological sample is subjected meets the adjusted threshold required for a positive test result, where subsequent prenatal test results are not generated for patients that fail to meet the adjusted threshold required for a positive test result for a prenatal test, thereby reducing the cost of identifying positive prenatal screen results compared to simultaneously generating test results for all of the prenatal tests in all of the patients.

Another embodiment of the invention is a method of reducing the number of unnecessary prenatal screening tests in a patient population, comprising: selecting a plurality of different prenatal tests comprising at least a first prenatal test and a second prenatal test for a prenatal screen; setting a minimum detection rate and a maximum false positive rate for the prenatal screen comprising the plurality of prenatal tests; adjusting the threshold required for a positive test result for the first prenatal test such that the false positive rate for the first prenatal test exceeds the maximum false positive rate for the prenatal screen; adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen; generating a first prenatal test result for biological samples from a plurality of patients; identifying a subset of biological samples that meets the adjusted threshold required for a positive test result for the first prenatal test and a subset of samples that do not meet the adjusted threshold required for a positive test result for the first prenatal test; generating a second prenatal test result for the subset of samples that meet the adjusted threshold required for a positive test result for the first prenatal test; determining whether the second prenatal test result meets the adjusted threshold required for a positive test result for the second prenatal test; sequentially subjecting samples from the subset of samples that meets the adjusted threshold required for a positive test result for the second prenatal test to testing in any remaining plurality of prenatal tests if the biological samples meet the adjusted threshold required for a positive test result for any subsequent prenatal test; and identifying patients as having a positive prenatal screen result if the test result for the final prenatal test to which their biological sample is subjected meets the adjusted threshold required for a positive test result, where subsequent prenatal test results are not generated for patients that fail to meet the adjusted threshold required for a positive test result for a prenatal test, thereby reducing the number of prenatal tests that must be generated to identify a positive prenatal screen result compared to simultaneously generating test results for all of the prenatal tests in all of the patients.

Another embodiment of the invention is a method of decreasing the risk of iatrogenic injury to a normal fetus, comprising: selecting a plurality of different prenatal tests comprising at least a first prenatal test and a second prenatal test for a prenatal screen; setting a minimum detection rate and a maximum false positive rate for the prenatal screen comprising the plurality of prenatal tests; adjusting the threshold required for a positive test result for the first prenatal test such that the false positive rate for the first prenatal test exceeds the maximum false positive rate for the prenatal screen; adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen; obtaining a biological sample from a patient identified as the source of a test result that meets the adjusted threshold required for a positive test result for the first prenatal test; generating a second prenatal test result for the biological sample; determining whether the second prenatal test result meets the adjusted threshold required for a positive test result for the second prenatal test; sequentially subjecting a biological sample from the patient to any remaining plurality of prenatal tests if the biological sample meets the adjusted threshold required for a positive test result for the second and any subsequent prenatal test; and identifying the patient as having a negative prenatal screen result if a test result of the biological sample fails to meet the adjusted threshold required for a positive test result for the second or any subsequent prenatal test; and identifying the patient as having a positive prenatal screen result if the test results for each of the plurality of prenatal tests to which a biological sample is subjected meets the adjusted threshold required for a positive test result for each of the plurality of prenatal tests, where the false positive rate for the prenatal screen at a given detection rate is less than the false positive rate for any of the plurality of tests alone, thereby improving the accuracy of information from the prenatal screen, and thereby reducing the number of women pregnant with a normal fetus advised to undergo an invasive prenatal procedure, decreasing the risk of iatrogenic injury to the fetus resulting from the invasive prenatal procedure.

Another embodiment of the invention is a method of increasing patient compliance in undergoing a recommended invasive prenatal procedure, comprising: selecting a plurality of different prenatal tests comprising at least a first prenatal test and a second prenatal test for a prenatal screen; setting a minimum detection rate and a maximum false positive rate for the prenatal screen comprising the plurality of prenatal tests; adjusting the threshold required for a positive test result for the first prenatal test such that the false positive rate for the first prenatal test exceeds the maximum false positive rate for the prenatal screen; adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen; generating a first prenatal test result for biological samples from a plurality of patients; identifying a subset of samples that meets the adjusted threshold for a positive test for the first prenatal test and a subset of samples that does not meet the adjusted threshold required for a positive test result for the first prenatal test; generating a second prenatal test result for the subset of samples that meet the adjusted threshold required for a positive test result for the first prenatal test; determining whether the second prenatal test result meets the adjusted threshold required for a positive test result for the second prenatal test; sequentially subjecting samples from the subset of samples that meet the adjusted threshold required for a positive test result for the second prenatal test to testing in any remaining plurality of tests if the biological samples meet the adjusted threshold required for a positive test result for any subsequent prenatal test; and identifying patients as having a positive prenatal screen result if the test result for the final prenatal test to which their biological sample is subjected meets the adjusted threshold required for a positive test result, where the false positive rate for the prenatal screen at a given detection rate is less than the false positive rate for any of the plurality of prenatal tests alone, and where a positive prenatal screen result leads to a more accurate recommendation for an invasive procedure, thereby increasing patient compliance in undergoing the invasive procedure recommended for a positive prenatal screen result.

Another embodiment of the invention is a method of screening for a fetal condition of interest, comprising: (a) obtaining a biological sample; (b) performing a first prenatal test on the biological sample; (c) detecting a positive or negative result for the first prenatal test; (d) reporting a negative test result if the biological sample generates a negative result for the first prenatal test; (e) performing a second prenatal test if the biological sample generates a positive result for the first prenatal test; (f) detecting a positive, negative, or inconclusive result for the second prenatal test; (g) reporting a negative test result if the biological sample generates a negative result for the second prenatal test; (h) reporting a positive test result if the biological sample generates a positive result for the second prenatal test; (i) reporting a positive test result for the first prenatal test and an inconclusive test result for the second prenatal test if the biological sample generates an inconclusive result for the second prenatal test; (j) optionally redrawing the biological sample if the biological sample generates an inconclusive result for the second prenatal test; and (k) optionally repeating steps (b) through (j) for the redrawn biological sample.

Another embodiment of the invention is a method of screening for a fetal condition of interest, comprising: (a) obtaining a biological sample; (b) performing a first prenatal test on the biological sample; (c) detecting a positive or negative result for the first prenatal test; (d) reporting a negative test result if the biological sample generates a negative result for the first prenatal test; (e) performing a second prenatal test if the biological sample generates a positive result for the first prenatal test; (f) detecting a positive, negative, or inconclusive result for the second prenatal test; (g) reporting a negative test result if the biological sample generates a negative result for the second prenatal test; (h) performing a third prenatal test if the biological sample generates a positive or inconclusive result for the second prenatal test; (i) reporting a negative test result if the biological sample generates a negative result for the third prenatal test; (j) reporting a positive test result if the biological sample generates a positive result for the third prenatal test; (k) optionally redrawing the biological sample if the biological sample generates an inconclusive result for the third prenatal test; and (l) optionally repeating steps (b) through (l) for the redrawn biological sample.

Another embodiment of the invention is a method of enriching a population of positive prenatal screen results for individuals carrying a fetus with a trait of interest. Another embodiment of the invention is a method of reducing the number of unnecessary invasive procedures recommended for a population.

In some embodiments of the invention, the invention comprises the following: a method where the adjusted threshold required for a positive test result for the second prenatal test is selected such that the integrated detection rate for the first and second prenatal tests is above the minimum detection rate for the prenatal screen, and such that the integrated false positive rate for the first and second prenatal tests is below the maximum false positive rate for the prenatal screen; a method where a third prenatal test is performed following the second prenatal test when the result of the second prenatal test meets the adjusted threshold required for a positive test result for the second prenatal test; a method where the third prenatal test is the final prenatal test in the prenatal screen; a method where a subsequent prenatal test is not performed in the prenatal screen when a biological sample fails to meet the adjusted threshold required for a positive test result in a prenatal test of the prenatal screen; a method where a third prenatal test result is not generated for at least one sample that does not meet the adjusted threshold required for a positive test result for the second prenatal test; a method where a third prenatal test is performed following the second prenatal test; and a method where the cost of the first prenatal test is lower than the cost of the second prenatal test; and a method where a positive prenatal screen result leads to a recommendation for an invasive procedure, thereby reducing the number of samples for which at least one additional non-invasive prenatal test is performed.

In some embodiments of the invention, the invention comprises the following: a method where the biological sample is plasma, serum, or whole blood; a method where the biological sample is from a human; a method where the first prenatal test or second prenatal test is a test for at least one biochemical marker; a method where the biochemical marker is selected from the group consisting of pregnancy-associated plasma protein A (PAPP-A), free beta human chorionic gonadotropin (Fβ-hCG), alpha-fetoprotein (AFP), human chorionic gonadotropin (hCG), unconjugated estriol (UE3), and dimeric inhibin A (DIA); a method where the first prenatal test or second prenatal test is a test for a fetal genetic variation in a cellular portion of the biological sample; a method where the first prenatal test or second prenatal test is a test for a fetal genetic variation in a cell-free nucleic acid portion of the biological sample; a method where the first prenatal test or second prenatal test is a test for a fetal genetic variation in a cellular portion of the biological sample; a method where the third prenatal test is a test for a fetal genetic variation in a cell-free nucleic acid portion of the biological sample; a method where the third prenatal test is a test for a fetal genetic variation in a cellular portion of the biological sample; a method where the first prenatal test comprises measuring the concentration level of at least two, three, or four markers; a method where the second prenatal test comprises measuring the concentration level of at least two, three, or four markers; a method where the third prenatal test comprises measuring the concentration level of at least two, three, or four markers; a method where the prenatal screening test is performed to detect a genetic variation; a method where the prenatal screening test is performed to detect a chromosomal copy number variation; a method where the chromosomal copy number variation is a trisomic or monosomic copy number variation; a method where the trisomic copy number variation is trisomy 21, trisomy 13, trisomy 18, trisomy 16, XXY, XYY, or XXX; a method where the monosomic copy number variation is monosomy X, monosomy 21, monosomy 22, monosomy 16, or monosomy 15; a method further comprising measuring an ultrasound marker of a chromosomal copy number variation in the subject from which the biological sample is obtained; a method where the ultrasound marker is nuchal translucency (NT) thickness or nuchal fold thickness; a method where the biological sample obtained is a portion of a sample taken from a subject prior to the first prenatal test; a method where the biological sample obtained is one of a plurality of samples taken from a subject prior to the first prenatal test; a method where the biological sample obtained is a portion of one of a plurality of samples taken from a subject following the first prenatal test; a method where the biological sample obtained is a portion of a sample taken from a subject following the first prenatal test; a method where the biological sample obtained is one of a plurality of samples taken from a subject following the first prenatal test; a method where the biological sample obtained is a portion of one of a plurality of samples taken from a subject following the first prenatal test; a method further comprising dividing each of the biological samples into at least a first portion and a second portion prior to the second prenatal test; a method where the second prenatal test is performed on the first portion of a divided sample; a method where the third prenatal test is performed on the second portion of a divided sample; a method where the cost charged for the first prenatal test is used to subsidize the cost for the second prenatal test; and a method where the prenatal screen is diagnostic.

In some embodiments of the invention, the invention comprises the following: a method where the false positive rate for the prenatal screen is less than 20%; a method where the false positive rate for the prenatal screen is less than 5%; a method where the detection rate for the prenatal screen is greater than 50%; and a method where the detection rate for the prenatal screen is greater than 80%.

In some embodiments, a method for screening and/or detecting a genetic condition in a fetus includes performing one of a plurality of tests prescribed by a reflex protocol, each of the tests including measuring a level of at least one biochemical marker indicative of a fetus with a genetic condition in a serum, plasma, whole blood, or urine sample obtained from a subject at one of a plurality of times during pregnancy. Each sequence of the reflex protocol begins with a first test (e.g., a biochemical marker measurement step) conducted on a first serum, plasma, whole blood or urine sample obtained from the subject within a first time during pregnancy. Each of the tests (e.g., fetal genetic variation detection steps) subsequent to the first test is selectively performed based on results from a precedent test, each sequence terminating in a respective final test conducted on serum, plasma, whole blood, or urine sampled from the subject at one of a plurality of different times subsequent to pregnancy. In addition to other clinical data, an indication of a subject's risk of having a fetus with a genetic condition or a diagnosis of a fetus with a genetic condition is provided for the individual based on the sequence of tests performed and on the results of the final test.

Embodiments provided herein also include methods and systems for assessing a subject's risk of having a fetus with a genetic condition. In some embodiments, a method for assessing a subject's risk of having a fetus with a genetic condition is provided, the method comprising the steps of: (a) measuring the level of a biochemical marker in a biological sample obtained from the subject; and (b) detecting a fetal genetic variation in a cell-free nucleic acid portion or a cellular portion of the biological sample from the subject if the level of the at least one biochemical marker in the sample of step (a) is indicative of a positive diagnosis of the genetic condition; thereby assessing the subject's risk of having a fetus with a genetic condition. In some embodiments, a method for assessing a subject's risk of having a fetus with a genetic condition is provided, the method comprising the steps of: (a) measuring the level of a biochemical marker in a biological sample obtained from the subject; (b) detecting a fetal genetic variation in a cell-free nucleic acid portion of the biological sample from the subject if the level of the biochemical marker in the sample of step (a) is indicative of a positive diagnosis of the genetic condition; and (c) detecting a fetal genetic variation in a cellular portion of the biological sample from the subject if the genetic variation detected in the sample of step (b) is indicative of a positive diagnosis of the genetic condition; thereby assessing the subject's risk of having a fetus with a genetic condition. In some embodiments, a method for assessing a subject's risk of having a fetus with a genetic condition is provided, the method comprising the steps of: (a) measuring the level of a biochemical marker in a biological sample obtained from the subject; (b) detecting a fetal genetic variation in a cellular portion of the biological sample from the subject if the level of the biochemical marker in the sample of step (a) is indicative of a positive diagnosis of the genetic condition; and (c) detecting a fetal genetic variation in a cell-free nucleic acid portion of the biological sample from the subject if the genetic variation detected in the sample of step (b) is indicative of a positive diagnosis of the genetic condition; thereby assessing the subject's risk of having a fetus with a genetic condition. In some embodiments, the method further comprises measuring an ultrasound marker of a chromosomal abnormality in the subject. In certain aspects, the ultrasound marker is nuchal translucency (NT) thickness or nuchal fold thickness.

Embodiments provided herein also include methods and systems for diagnosing a genetic condition in a fetus. In some embodiments, a method for detecting a genetic condition in a fetus of a subject is provided, the method comprising the steps of: (a) measuring the level of a biochemical marker in a biological sample obtained from the subject; and (b) detecting a fetal genetic variation in a cell-free nucleic acid portion or a cellular portion of the biological sample from the subject if the level of the at least one biochemical marker in the sample of step (a) is indicative of a positive diagnosis of the genetic condition; thereby detecting a genetic condition in a subject. In some embodiments, a method for detecting a genetic condition in a fetus of a subject is provided, the method comprising the steps of: (a) measuring the level of at least one biochemical marker in a biological sample obtained from the subject; (b) detecting a fetal genetic variation in a cell-free nucleic acid portion of the biological sample from the subject if the level of the biochemical marker in the sample of step (a) is indicative of a positive diagnosis of the genetic condition; and (c) detecting a fetal genetic variation in a cellular portion of the biological sample from the subject if the genetic variation detected in the sample of step (b) is indicative of a positive diagnosis of the genetic condition; thereby detecting a genetic condition in a subject. In some embodiments, a method for detecting a genetic condition in a fetus of a subject is provided, the method comprising the steps of: (a) measuring the level of a biochemical marker in a biological sample obtained from the subject; (b) detecting a fetal genetic variation in a cellular portion of the biological sample from the subject if the level of the biochemical marker in the sample of step (a) is indicative of a positive diagnosis of the genetic condition; and (c) detecting at least one fetal genetic variation in a cell-free nucleic acid portion of the biological sample from the subject if the genetic variation detected in the sample of step (b) is indicative of a positive diagnosis of the genetic condition; thereby detecting a genetic condition in a subject. In some embodiments, the method further comprises measuring an ultrasound marker of a chromosomal abnormality in the subject. In certain aspects, the ultrasound marker is nuchal translucency (NT) thickness or nuchal fold thickness.

In some embodiments, the biological sample is plasma, serum, whole blood, or urine. In other aspects, the subject is a human subject. More particularly, the subject is a pregnant female human. In yet other aspects, the biochemical marker measured in the methods described herein is selected from the group consisting of Pregnancy-associated plasma protein A (PAPP-A), free beta human chorionic gonadotropin (Fβ-hCG), alpha-fetoprotein (AFP), human chorionic gonadotropin (hCG), unconjugated estriol (uE), and dimeric inhibin A (DIA). In some embodiments, step (a) of the method comprises measuring the concentration level of at least two, three, or four biochemical markers. In other aspects, the genetic condition is a chromosomal variation. In further aspects, the chromosomal variation is a trisomic or monosomic copy number variation. In yet further aspects, the trisomic copy number variation is trisomy 21, trisomy 13, trisomy 18, trisomy 16, XXY, XYY, or XXX. In other aspects, the monosomic copy number variation is monosomy X, monosomy 21, monosomy 22, monosomy 16, or monosomy 15.

### BRIEF DESCRIPTION OF THE FIGURES

FIG. 1 is a flowchart illustrating a reflex protocol for screening and/or diagnosing a fetal genetic condition in a biological sample in accordance with an embodiment provided herein.

FIG. 2 is a flowchart illustrating a reflex protocol for screening and/or diagnosing a fetal genetic condition in a biological sample in accordance with an embodiment provided herein.

### DESCRIPTION OF THE INVENTION

Before the present systems and methods are described, it is to be understood that the invention is not limited to the particular methodologies, protocols, assays, and reagents described, as these may vary.

It must be noted that as used herein and in the appended claims, the singular forms “a,” “an,” and “the” include plural references unless context clearly dictates otherwise. Thus, for example, a reference to “a genetic condition” may include a plurality of such conditions; a reference to a “fetal genetic variation” may be a reference to one or more fetal genetic variations, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, systems, and materials are now described. All publications cited herein are incorporated herein by reference in their entirety, and for the purpose of describing and disclosing the methodologies, reagents, and tools, etc. reported in the publications that might be used in connection with the invention or which are specifically referenced herein. Nothing herein is to be construed as an admission that the invention is not entitled to antedate such disclosure, for example by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

The section headings are used herein for organizational purposes only, and are not to be construed as in any way limiting the subject matter described herein.

### Statistical Measures

### Genetic Variation Test

A two-class genetic variation test has two possible results. A positive test result indicates that an individual exhibits or is likely to exhibit a genetic condition of interest and a negative test result indicates that an individual does not exhibit or is not likely to exhibit the genetic condition of interest. As such, the reliability of a genetic test is related to how often the result of the test correctly identifies an individual as “positive” or “negative” for the genetic condition. True positives (TP) and true negatives (TN) are test results that accurately identify individuals as positive (e.g. “affected”) and negative (e.g. “unaffected”), respectfully. A false positive (FP) is a test result that incorrectly classifies an individual as a positive when they are in fact negative for the genetic condition. Likewise, a false negative (FN) is a test result that incorrectly classifies an individual as a negative when they are in fact positive for the genetic condition. Measures of TP, TN, FP and FN are used to calculate the sensitivity, specificity, PPV and NPV for a genetic variation test.

### Sensitivity/Detection Rate

The “sensitivity” or “detection rate” of a test is a measure of the ability of the test to correctly identify an affected individual, or an individual who will develop the genetic condition of interest. The closer the sensitivity is to one, the more accurate the test is in identifying affected individuals. Specifically, the sensitivity refers to the proportion of affected individuals who are correctly diagnosed as such by the test, and is calculated as the number of individuals correctly identified as affected (TP) divided by the total number of affected individuals (TP+FN). A high sensitivity is preferred so that most affected individuals are identified as such by the genetic test.

### Specificity/False Positive Rate

The “specificity” or “false positive rate” of a test is a measure of the ability of the test to correctly identify an unaffected individual, or an individual who will not develop the genetic condition of interest. The closer the specificity is to one, the more accurate the test is in identifying unaffected individuals. Specifically, the specificity refers to the proportion of unaffected individuals who are correctly identified as such by the test, and is calculated as the number of individuals correctly identified as unaffected (TN) divided by the total number of unaffected individuals (TN+FP). A false positive rate is calculated as 1 minus the specificity (1−((TN)/(TN+FP))). A high specificity is preferred so that the number of individuals who are incorrectly identified as affected is minimized. Thus, for a given risk cutoff value, the sensitivity is calculated as the proportion of case individuals with a score higher than the risk cutoff value, and the specificity is calculated as the proportion of control individuals with a score lower than or equal to the risk cutoff value (or, one minus the proportion of control individuals with a score higher than the risk cutoff value).

### Predictive Values

The “positive predictive value” (PPV) of a genetic test assesses the reliability of a positive test outcome/result, and is computed as the proportion of people with a positive test result who actually have the genetic condition of interest. In other words, it is the probability that a positive test result accurately identifies an individual who has the genetic condition, and is calculated as the number of individuals correctly identified as affected (TP) divided by the total number of individuals identified as affected by the genetic test (TP+FP). In many cases, a high PPV is preferred so that most individuals who are identified as affected are actually affected. For example, a PPV of 0.98 means that an individual with a positive test result has a 98% chance of having or developing the genetic condition.

The “negative predictive value” (NPV) of a genetic test assesses the reliability of a negative test outcome/result, and is computed as the proportion of people with a negative test result who do not have the genetic condition of interest. Put another way, it is the probability that a negative test result accurately identifies an individual who does not have the genetic condition, and is calculated as the number of individuals correctly identified as unaffected (TF) divided by the total number of individuals identified as unaffected (TN+FN). A high NPV is sometimes preferred so that most individuals who are identified as unaffected are actually unaffected (e.g., in excluding subjects at risk for adverse events associated with the administration of a specific drug). For example, an NPV of 0.999 means that an individual with a negative test result has only a 0.1% chance of having or developing the genetic condition (e.g., of experiencing the adverse event in response to the drug). Thus, for a given risk cutoff value, the PPV may be calculated as the proportion of all individuals with a score higher than the risk cutoff value that are actually in the case group, and the NPV is calculated as the proportion of all individuals with a score lower than or equal to the risk cutoff value that are actually in the control group.

### Receiver Operating Characteristics (ROC)

In some embodiments, the specificity and/or sensitivity of one or more tests described herein is modified by varying the threshold of the Receiver Operating Characteristics (ROC) curves of the one or more tests. ROC curves provide information as to the number of true positives (TP) and true negatives (TN), as well as the number of false positives (FP) and false negatives (FN) for a given test when varying thresholds are applied. Based on the ROC curves, the sensitivity of a test can be calculated as the probability that the test correctly classifies a positive result, wherein: Sensitivity=TP/(TP+FN). Similarly, the ROC curve also identifies the specificity of a test, defined as the probability that the test correctly classifies a negative result, wherein: Specificity=TN/(TN+FP). An ROC curve can be generated by plotting the sensitivity over (1−the specificity) for varying thresholds. (Nielsen, N. E. et al., (2000) J. Intern. Med. 247:43-52). In some embodiments, the threshold of the ROC curve for one or more test used in the reflex protocols provided herein is modified to obtain higher sensitivity (i.e., increase detection).

### Integrated Reflex Protocols

The embodiments described herein provide method and systems for screening and/or detecting a genetic condition in a fetus according to various reflex protocols of biochemical marker measurement steps and fetal genetic variation detection steps in which subsequent biochemical marker measurement steps and/or fetal genetic variation detection steps are selectively performed based on the results of previous biochemical marker measurements or fetal genetic variation detection and without the need for human decision making.

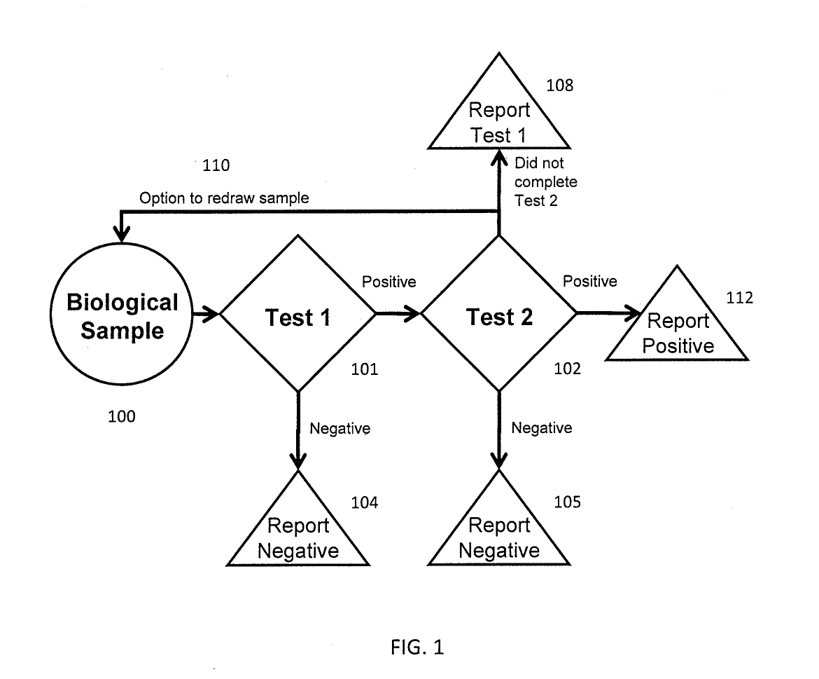

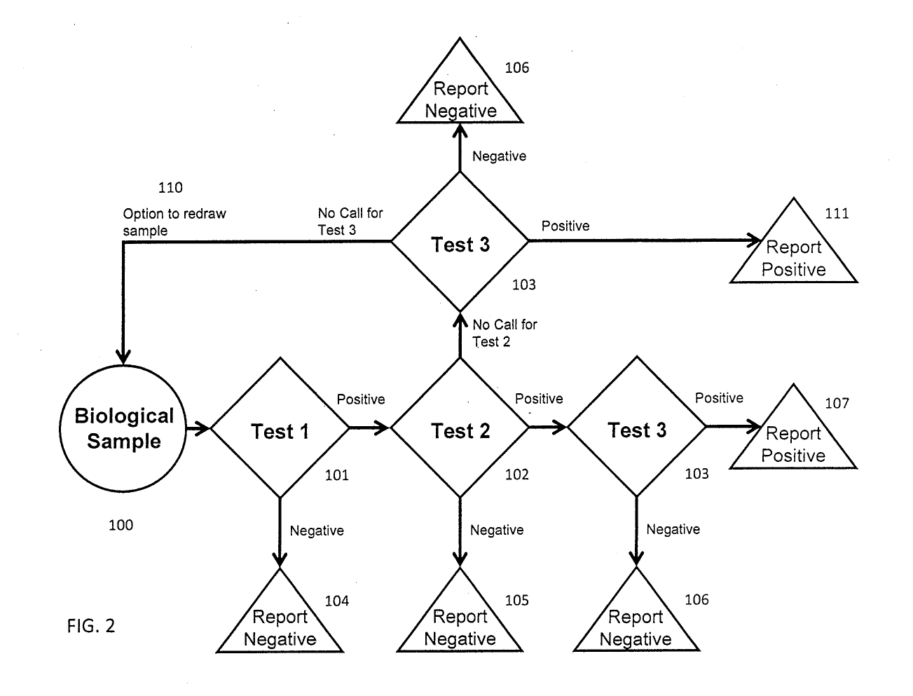

Referring generally to FIGS. 1 and 2, operational flow diagrams are shown for an integrated reflex protocol. The depicted reflex protocols represent an organization of tests or assays for measuring biochemical markers and/or detecting fetal genetic variations in a biological sample. More specifically, the illustrative integrated reflex protocol of FIGS. 1 and 2 employ tests for biochemical marker measurements including, for example, immunochemistry and clinical chemistry assays and tests for fetal genetic variation detection including, for example, fetal cell-based or cell-free fetal nucleic acid assays.

For clarity, as will be more fully understood herein, as depicted in FIGS. 1 and 2, the circle shapes indicate a biological sample to be examined in a reflex protocol as described herein, square shapes indicate assay execution steps (e.g., one or more biochemical marker measurements), and triangle shapes indicate a report of the test results (e.g., sample is positive or negative for a fetal genetic condition) which lead to an endpoint. In addition, for simplicity, reference to a biological sample or a portion thereof, is generically used to refer to using whole blood, serum, plasma, or urine as appropriate for the assay or test conducted.

Referring now more specifically to FIG. 1, upon obtaining a biological sample (100) from a subject with a fetus, a first screening or diagnostic test (step 101) is performed on a first portion of the biological sample. In some embodiments, the first screening or diagnostic test can be an assay for measuring a biochemical marker or an assay for detecting a fetal genetic variation. If in step 101, the first test produces a positive result (e.g., the level of a measured biochemical marker is above or below some threshold level), indicating increased risk or presence of a fetal genetic condition, then a second screening or diagnostic test (step 102) is performed using a second portion of the biological sample. Alternatively, if in step 101 the test result is negative (e.g., the level of a measured biochemical marker is normal or within a threshold range), then the negative result is reported (step 104) leading to an endpoint.

If in step 102, the second test produces a positive result (e.g., a fetal genetic variation is detected in a cell-free nucleic acid portion), indicating increased risk or presence of a fetal genetic condition, then the positive result is reported (step 112) leading to an endpoint. Alternatively, if in step 102 the test result is negative (e.g., no fetal genetic variation is detected in a cell-free nucleic portion), then the negative result is reported (step 105) leading to an endpoint. Alternatively, if in step 102 the test result is inconclusive (i.e., no call is made), then the positive result of the first test of step 101 is reported (step 108). Alternatively, if in step 102 the test result is inconclusive (i.e., no call is made), then an option to redraw a biological sample is suggested (step 110). In some embodiments, the second screening or diagnostic test can be an assay for measuring a biochemical marker or an assay for detecting a fetal genetic variation. In some embodiments, further diagnostic testing (e.g., amniocentesis) is suggested following a report of a positive result in step 112.

Referring now more specifically to FIG. 2, upon obtaining a biological sample (100) from a subject with a fetus, a first screening or diagnostic test (step 101) is performed on a first portion of the biological sample. In some embodiments, the first screening or diagnostic test can be an assay for measuring a biochemical marker or an assay for detecting a fetal genetic variation. If in step 101, the first test produces a positive result (e.g., the level of a measured biochemical marker is above or below some threshold level), indicating increased risk or presence of a fetal genetic condition, then a second screening or diagnostic test (step 102) is performed using a second portion of the first biological sample. Alternatively, if in step 101 the test result is negative (e.g., the level of a measured biochemical marker is normal or within a threshold range), then the negative result is reported (step 104) leading to an endpoint.

If in step 102, the second test produces a positive result (e.g., a fetal genetic variation is detected in a cell-free nucleic acid portion), indicating increased risk or presence of a fetal genetic condition, then a third screening or diagnostic test (step 103) is performed using a third portion of the first biological sample. Alternatively, if in step 102 the test result is negative (e.g., no fetal genetic variation is detected in a cell-free nucleic portion), then the negative result is reported (step 105) leading to an endpoint. Alternatively, if in step 102 the test result is inconclusive (i.e., no call is made), then a third screening or diagnostic test (step 103) is performed using a third portion of the first biological sample. In some embodiments, the second screening or diagnostic test can be an assay for measuring a biochemical marker or an assay for detecting a fetal genetic variation.

If in step 103, the third test produces a positive result (e.g., a fetal genetic variation is detected in a cellular portion of the biological sample), indicating increased risk or presence of a fetal genetic condition, then the positive result is reported (step 107 or step 111) leading to an endpoint. In some embodiments, the third screening or diagnostic test can be an assay for measuring a biochemical marker or an assay for detecting a fetal genetic variation. Alternatively, if in step 103 the test result is negative (e.g., no fetal genetic variation is detected in a cellular portion of the biological sample), then the negative result is reported (step 106) leading to an endpoint. Alternatively, if in step 103 the test result is inconclusive (i.e., no call is made), then an option to redraw a biological sample is suggested (step 110). In some embodiments, further diagnostic testing (e.g., amniocentesis) is suggested following a report of a positive result in step 107 or step 111.

It is appreciated that the illustrative embodiments of FIGS. 1 and 2 provide various pathways (i.e., a pathway representing a series of tests performed according to a reflex method as described herein) for improved screening and/or detection of fetal genetic conditions as represented by the diagnostic endpoint indicative of a fetal genetic condition (e.g., step 107) which result from biochemical marker measurements and detection of fetal genetic variations in various portions of a biological sample from a subject according to a reflex protocol provided herein.

In some embodiments, the first test used in the reflex protocol is not the same as the second test used in the reflex protocol. As a non-limiting example, in carrying out the methods described herein, the first test used in the reflex protocol can be a biochemical marker test, in which case the second test used in the reflex protocol is a cell-free fetal nucleic acid test or a fetal cell nucleic acid test. Alternatively, if the first test used in the reflex protocol is a cell-free fetal nucleic acid test, then the second test used in the reflex protocol can be a fetal cell nucleic acid test or a biochemical marker test. Additionally, if the first test used in the reflex protocol is a fetal cell nucleic acid test, then the second test used in the reflex protocol can be a cell-free fetal nucleic acid test or a biochemical marker test. Briefly, the order for using a biochemical marker test or fetal nucleic acid test to perform the reflex testing is interchangeable.

The cost of each test used in the reflex protocol may be considered in selecting the order of tests. Such costs include, but are not limited to, laboratory expenses (consumables and staff), genetic counseling for subjects, service costs (including processing results and monitoring the service), overheads, and training. Cost-effectiveness analysis can also be utilized to determine the order of tests in the reflex protocols (Caughey (2005) Gynecol Obstet Invest. 60:11-8). One of skill in the art will readily appreciate that as the cost of a given test decreases over time, e.g., the cost of nucleic acid sequencing decreases, the selection of the order of tests used in the reflex protocol will vary. For example, sequencing may be selected for the first test in a prenatal screen if a sequencing assay becomes more cost effective than a biochemical assay that is selected for the second test in a prenatal screen.

In some embodiments, the threshold of the first test is adjusted such that the false positive rate for the first test exceeds the maximum false positive rate for the prenatal screen. In some embodiments, the threshold of more than one test is adjusted such that the false positive rate for these tests exceeds the maximum false positive rate for the prenatal screen. For example, in some embodiments, the threshold of the first and second tests are adjusted such that the false positive rate for the first and second tests each exceed the maximum false positive rate for the prenatal screen.

In some embodiments, the false positive rate for a prenatal screen is less than, or less than about, 20%. In a preferred embodiment, the false positive rate for a prenatal screen is less than, or less than about, 5%. In some embodiments, the false positive rate for a prenatal screen is, is about, is not more than, is not more than about 20%, 19%, 18%, 17%, 16%, 15%, 14%, 13%, 12%, 11%, 10%, 9%, 8%, 7%, 6%, 5%, 4%, 3%, 2%, 1%, or a range defined by any two of the preceding values. In some embodiments, the prenatal screen is a screening test. In some embodiments, the prenatal screen is diagnostic.

In some embodiments, the detection rate for a prenatal screen is greater than about 50%. In a preferred embodiment, the detection rate for a prenatal screen is greater than about 80%. In some embodiments, the detection rate for a prenatal screen is, is about, is at least, is at least about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a range defined by any two of the preceding values.

In some embodiments, only a single test is performed by a particular entity to complete a prenatal screen. For example, in some embodiments, information is obtained for a first test performed by a first entity, and a second test is performed by a second entity to complete the prenatal screen. In some embodiments, multiple tests are performed by a particular entity for a prenatal screen. For example, in some embodiments, information is obtained for a first test performed by a first entity, and a plurality of tests are performed by a second entity to complete the prenatal screen. In some embodiments, a plurality of tests are performed by a second entity without any prior information.

The embodiments provided herein contemplate that a patient may have additional screening tests performed. In some embodiments, the first test for the purposes of a prenatal screen provided herein is not the first test run on a patient with respect to prenatal testing. In some embodiments, the final test for the purposes of a prenatal screen provided herein is not the final test run on a patient with respect to prenatal testing.

As used herein, the term “screen” is not meant to limit the type of or number of test(s) or sample(s) that can be used in connection with the methods described herein. For example, in some embodiments, a prenatal screen is performed for a single sample. In some embodiments, a prenatal screen is performed for a plurality of samples. Further, in some embodiments, a prenatal screen provides a risk estimate for a condition of interest. In some embodiments, the prenatal screen is diagnostic for a condition of interest.

As used herein, the term “sequential testing” is not meant to imply any reporting of test results. For example, in some embodiments, results are not provided until a prenatal screen has been completed. In some embodiments, results are provided after each prenatal screen test. In some embodiments, only positive test results are provided after a particular test or number of tests have been performed. In some embodiments, only negative test results are provided after a particular test or number of tests have been performed.

### Bayesian Inference

In some embodiments, Bayesian inference analysis is used in the methods and systems provided herein. Bayesian inference analysis takes into account pre-test probability (whether subjectively determined or via an assay) to determine the predictive values of the subsequent test. Bayesian inference analysis can be used to systematically enrich the subject population for true positive (e.g., “affected”) subjects, reduce false positive subjects, or to enhance the accuracy of diagnosis in the reflex protocols provided herein.

### Prenatal Screening

The prenatal screens described herein may include any relevant prenatal test known to one of skill in the art. In some embodiments, the prenatal screens comprise a single prenatal test. In some embodiments, the prenatal screens comprise a plurality of prenatal tests. In some embodiments, the prenatal tests are screening tests. In some embodiments, the prenatal tests are diagnostic tests.

In some embodiments, each test in a prenatal screen is a biochemical assay performed using a blood sample. In some embodiments, each test in a prenatal screen is a test for a genetic variation using the cell-free portion of a blood sample. In some embodiments, each test in a prenatal screen in a test for a genetic variation using a cellular portion of a blood sample. In some embodiments, the tests in a prenatal screen are performed using a combination of plasma, serum, cellular, or cell-free portions of a blood sample or samples. For example, in some embodiments, the first test is performed in the plasma portion of a blood sample from a patient, and the second test is performed in the cellular portion of the same blood sample. In some embodiments, the first test is performed in the plasma portion of a first blood sample from a patient, and the second test is performed in the cellular portion of a second blood sample from the patient. In some embodiments, the first test is performed in a whole blood or serum portion of a first blood sample from a patient.

In some embodiments, each test in a prenatal screen is performed using a sample or samples obtained during the first trimester. In some embodiments, the first test of a prenatal screen is performed using a sample obtained during the first trimester, and the subsequent test or tests are performed in a sample or samples obtained during the second trimester. In some embodiments, each test in a prenatal screen is performed using a sample or samples obtained during the second trimester.

In some embodiments, the prenatal screen includes a biochemical assay for a first test, a plasma-based biochemical assay for a second test, a cell-based biochemical assay as a third test, and an invasive procedure as a fourth test. For example, in some embodiments, the first test is a screen comprising PAPP-A and hCG measurements; the second test is a penta screen comprising AFP, hCG, uE3, DIA, and invasive trophoblast antigen (ITA) measurements (i.e., Maternal Serum Screen 5, Quest Diagnostics, Madison, N.J.); the third test is a test for a genetic variation in a cellular portion of a sample; and the fourth test is amniocentesis. In some embodiments, the biochemical assays further comprise a non-biochemical variable, such as maternal age or an ultrasound marker measurement. For example, in some embodiments, the first test is an integrated screening comprising PAPP-A, hCG, and nuchal translucency measurements. Alternatively, in some embodiments, the first test is a non-biochemical test. For example, in some embodiments, the first test consists of an ultrasound marker measurement.

In some embodiments, the prenatal screen includes biochemical assays for a first test, cell-based biochemical assays for a second test, and an invasive procedure as a third test. For example, in some embodiments, the first test is a quad screen comprising AFP, hCG, uE3, and inhibin measurements; the second test is for a genetic variation in a cellular portion of a sample; and the third test is amniocentesis.

In some embodiments, the prenatal screen includes a genetic screen for a first test and a genetic diagnostic for a second test. In some embodiments, the first test is performed using a plasma portion of a blood sample from a patient, and the second test is performed using a cellular portion of the same blood sample. In some embodiments, the first test is performed using the plasma portion of a first blood sample from a patient, and the second test is performed using a cellular portion of a second blood sample from the same patient. In some embodiments, the first test further comprises a nuchal translucency measurement.

In some embodiments, the prenatal screen includes a biochemical screen for a first test and a genetic diagnostic for a second test. For example, in some embodiments, the first test is a screen comprising PAPP-A and Fβ-hCG measurements performed using a blood sample, and the second test is a diagnostic test for a genetic variation performed using a cellular portion of a blood sample. In some embodiments, the first test is a screen comprising PAPP-A and Fβ-hCG measurements performed using a blood sample, and the second test is a diagnostic test for a genetic variation performed using a cell-free portion of a blood sample. In some embodiments, the same blood sample is used for the first and second tests. In some embodiments, a different blood sample is used for the first and second tests. In some embodiments, the first test is or further comprises a nuchal translucency measurement.

In some embodiments, the prenatal screen includes a plasma-based biochemical screen for a first test and a cell-based biochemical screen for a second test. For example, in some embodiments, the first test is for a genetic variation in a plasma sample (e.g., a maternal plasma sample); and the second test is for a genetic variation in a cellular portion of a sample.

### Biological Samples

One or more samples may be used in the systems and methods described herein. In some embodiments, only a single biological sample is used. In some embodiments, a sample contains only fetal genetic material. In some embodiments, a sample contains an admixture of fetal and maternal genetic material. As used herein, the term “sample” can be used interchangeably with the term “biological sample.”

In some embodiments, the sample is a maternal blood sample. A maternal blood sample can be separated into various portions including, for example, a cellular portion comprising a mixture of maternal and fetal cells or a cell-free nucleic acid portion comprising mixed maternal and fetal cell-free nucleic acids. In some embodiments, the cell-free nucleic acid portion comprising mixed maternal and fetal cell-free nucleic acids is present in the plasma or serum portion of the maternal blood sample. In some embodiments, a serum or plasma portion of the maternal blood sample is used for measuring a biochemical marker as described herein. In some embodiments, about 20-40 mL of blood is drawn from a pregnant woman. Blood samples can be collected at any point during pregnancy. For example, in some embodiments, the maternal sample is collected during the first trimester. In some embodiments, the maternal sample is collected during the second trimester. In some embodiments, blood is drawn at 10-18 weeks gestational age. However, blood can be drawn earlier in the pregnancy or after 18 weeks gestational age. The time of collection may vary depending on the information sought or the standards of prenatal care. Blood samples can also be collected at any time during the day. In some embodiments, blood is collected in the morning. In some embodiments, blood is collected in the afternoon.

Blood can be drawn from any suitable area of the body, including an arm, a leg, or blood accessible through a central venous catheter. In some embodiments, blood is collected following a treatment or activity. For example, blood can be collected following a pelvic exam. The timing of collection can also be coordinated to increase the number of fetal cells or cell-free nucleic acids present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, or a DNA or RNA preservative following collection. In some embodiments, blood is collected via venipuncture using vacuum collection tubes containing an anticoagulant such as EDTA or heparin. Blood can also be collected using a heparin-coated syringe and hypodermic needle. Blood can also be combined with components that will be useful for cell culture. For example, in some embodiments, blood is combined with cell culture media or supplemented cell culture media (e.g., cytokines).

Maternal samples can also be obtained from other sources known in the art, including serum, plasma, urine, cervical swab, tears, saliva, buccal swab, skin, or other tissues. Samples of mixed maternal and fetal cells and samples of mixed maternal and fetal cell-free nucleic acids can also be obtained from other sources known in the art, including serum, plasma, urine, cervical swab, cervical lavage, uterine lavage, culdocentesis, lymph node, or bone marrow. For example, in some embodiments, the source of a sample of mixed maternal and fetal cell-free nucleic acids is a cervical swab. In some embodiments, the fetal cell-free nucleic acids comprise DNA. In another embodiment, the fetal cell-free nucleic acids comprise RNA or cDNA. As used herein, nucleic acid means a deoxyribonucleic acid (e.g., DNA, mtDNA, gDNA, or cDNA), ribonucleic acid (e.g., RNA or mRNA), or any other variant of nucleic acids known in the art.

As used herein, a “sample” can refer to a single specimen/measurement obtained at a particular time (e.g., a single blood draw or ultrasound measurement during a single office visit), or multiple specimens/measures obtained at a particular time (e.g. three separate blood draws at a single office visit). A “sample” can also refer to a single specimen which is later divided (e.g., a blood draw divided into multiple tubes, or fractionated into various components). A “sample” can also refer to multiple specimens obtained at different time points (e.g., a blood draw at 15 weeks gestation, and a second blood draw obtained at 20 weeks gestation). In some embodiments, a sample is obtained from an individual on a single day. In other embodiments, the sample is obtained from the individual on different days.

It will be recognized by one of skill in the art that the biological samples described herein can be collected from any appropriate source at any appropriate time. In some embodiments, a single biological sample is used for multiple prenatal tests. For example, in some embodiments, a blood sample collected from a patient is divided and used for a first and a second prenatal test. In some embodiments, multiple biological samples are collected from a patient for use in multiple prenatal tests. For example, in some embodiments, separate blood samples are collected from a patient for a first and a second prenatal test. In some embodiments, different biological samples are collected from a patient for use in multiple prenatal tests. For example, a blood sample can be collected from a patient for a first prenatal test, and a urine sample can be collected from the patient for a second prenatal test. Alternatively, a blood sample can be collected from a patient, divided, and used for a first prenatal test. If the first prenatal test is positive, a urine sample can be collected from the patient for a second prenatal test. If the second prenatal test is positive, a remaining portion of the divided blood sample can be used for a third prenatal test.

### Biochemical Markers

Beta Human Chorionic Gonadotropin (β-hCG)

Beta human chorionic gonadotropin (β-hCG) and free beta human chorionic gonadotropin (Fβ-hCG) are recognized biomarkers for detecting genetic conditions (e.g., Down syndrome) in biological samples. U.S. Pat. Nos. 5,324,668, 5,100,806, 5,252,489, 5,258,907, 5,316,953, 5,324,667 disclose the measurement of Fβ-hCG in the blood of pregnant women for detecting a fetus with aneuploidy. U.S. Pat. No. 6,025,149 discloses the measurement of Fβ-hCG in the urine of pregnant women for detecting a fetus with aneuploidy. In some embodiments, the level of Fβ-hCG or β-hCG in a biological sample is measured by immunological methods which can include immunoassay techniques and other techniques known in the art. In some embodiments, the level of Fβ-hCG or β-hCG is then compared to a set of reference data to indicate a positive diagnosis of a genetic condition.

In the middle to late second trimester, the levels of β-hCG can be used in conjunction with the levels of AFP to screen for chromosomal abnormalities, in particular for Down syndrome. An elevated β-hCG level coupled with a decreased AFP level suggests Down syndrome. In some embodiments, a β-hCG/β-hCG screen, optionally in conjunction with an AFP screen, is the first, second, third or a subsequent test in the prenatal screen/diagnosis disclosed herein.

### Pregnancy-Associated Plasma Protein A (PAPP-A)

Pregnancy-associated plasma protein A, pappalysin 1, also known as PAPP-A, is a protein biomarker used in screening tests for fetal genetic conditions. (See, e.g., Ndumbe at al. (2008) Obstet Gynecol Surv. 63:317-28). For example, low plasma level of PAPP-A can indicate an increased risk of having a fetus with aneuploidy. In some embodiments, the level of PAPP-A in a biological sample is measured by immunological methods which can include immunoassay techniques and other techniques known in the art. In some embodiments, the level of PAPP-A is then compared to a set of reference data to indicate a positive diagnosis of a genetic condition. In some embodiments, a PAPP-A screen is the first, second, third or a subsequent test in the prenatal screen/diagnosis disclosed herein.

### Alpha-Fetoprotein (AFP)

The developing fetus has two major blood proteins—albumin and alpha-fetoprotein (AFP). The mother typically has only albumin in her blood, and thus, the AFP test can be utilized to determine the levels of AFP from the fetus. Ordinarily, only a small amount of AFP gains access to the amniotic fluid and crosses the placenta to mother's blood. However, if the fetus has a neural tube defect, then more AFP escapes into the amniotic fluid. Neural tube defects include anencephaly (failure of closure at the cranial end of the neural tube) and spina bifida (failure of closure at the caudal end of the neural tube). The incidence of such defects is about 1 to 2 births per 1000 in the United States. Also, if there are defects in the fetal abdominal wall, the AFP from the fetus will end up in maternal blood in higher amounts.

The amount of AFP in maternal blood increases with gestational age, and thus for the AFP test to provide accurate results, the gestational age must be known. Also, the race of the mother and presence of gestational diabetes can influence the maternal blood level of AFP that is to be considered normal. Maternal blood AFP levels are typically reported as multiples of the mean (MoM). The greater the MoM, the more likely a defect is present. The AFP test has greater sensitivity between 16 and 18 weeks gestation, but can be used between 15 and 22 weeks gestation. Maternal blood AFP levels tend to be lower when chromosomal abnormalities are present. In some embodiments, an AFP screen is the first, second, third or a subsequent test in the prenatal screen/diagnosis disclosed herein.

### Estriol

The amount of estriol in maternal serum is dependent upon a viable fetus, a properly functioning placenta, and maternal well-being. Dehydroepiandrosterone (DHEA) is made by the fetal adrenal glands, and is metabolized in the placenta to estriol. The estriol enters the maternal circulation and is excreted by the maternal kidney in urine or by the maternal liver in the bile. Normal levels of estriol, measured in the third trimester, will give an indication of general well-being of the fetus. If the estriol level drops, then the fetus is threatened and an immediate delivery may be necessary. Estriol tends to be lower when Down syndrome is present and when there is adrenal hypoplasia with anencephaly. In some embodiments, an estriol screen is the first, second, third or a subsequent test in the prenatal screen/diagnosis disclosed herein.

### Inhibin A

Inhibin A is a protein secreted by the placenta and the corpus luteum during pregnancy and can be measured in maternal serum. Inhibin A is an accepted biomarker for prenatal screening. (Aitkin (1996) N Engl J Med. 334:1231-6). An increased level of inhibin A is associated with an increased risk for chromosomal abnormality (e.g., Down syndrome). (See, e.g., Wald et al. (1996) Prenatal Diagnosis. 16:143-153). In some embodiments, an inhibin A screen is the first, second, third or a subsequent test in the prenatal screen/diagnosis disclosed herein.

### Triple Screen Test

The triple screen test is a non-invasive prenatal screening test used to identify increased risk of a fetus with a genetic condition. The triple screen test comprises analysis of maternal serum alpha-feto-protein (AFP), human chorionic gonadotrophin (hCG), and unconjugated estriol (uE3). The blood test is usually performed 16-18 weeks after the last menstrual period. While the triple screen test is non-invasive, abnormal test results are not indicative of a birth defect. Rather, the test only indicates an increased risk and suggests that further testing is needed. For example, 100 out of 1,000 women will have an abnormal result from the triple screen test. However, only 2-3 of the 100 women will have a fetus with a birth defect. Use of a triple screen test is specifically contemplated in embodiments of the systems and methods provided herein. In some embodiments, the “triple screen” is the first, second, third or a subsequent test in the prenatal screen/diagnosis disclosed herein.

### Quad Screen Test

The quad screen test is a second trimester aneuploidy risk assessment which includes four biochemical markers, AFP, total hCG, estriol and inhibin A. The test is performed between 15 and 18 weeks of pregnancy (but between 14 and 22 weeks is possible). The quad screen detects 80% of Down syndrome pregnancies and includes screening for neural tube defects. Use of a quad screen test is specifically contemplated in embodiments of the systems and methods provided herein. In some embodiments, the “quad screen” is the first, second, third or a subsequent test in the prenatal screen/diagnosis disclosed herein.

### Penta Screen Test

The penta screen test is a second trimester prenatal screening test used to identify increased risk of a fetus with neural tube defects, Down syndrome, and trisomy 18 (Maternal Serum Screen 5, Quest Diagnostics, Madison, N.J.). The penta screen test comprises analysis of maternal serum AFP, hCG, uE3, DIA, and invasive trophoblast antigen (ITA). The penta screen detects 83% of Down syndrome pregnancies. Use of the penta screen test is specifically contemplated in embodiments of the systems and methods provided herein. In some embodiments, the “penta screen” is the first, second, third or a subsequent test in the prenatal screen/diagnosis disclosed herein.

### Non-Biochemical Markers

### Ultrasound

Ultrasound is a non-invasive procedure that has been used routinely for prenatal screening of fetal genetic conditions. (See, e.g., Tamsel et al (2007) Diagn Intery Radiol. 13:97-100). High frequency sound waves are used to generate visible images from the pattern of the echoes made by different tissues and organs, including the fetus in the amniotic cavity. The developing embryo can be visualized at about 6 weeks of gestation. The major internal organs and extremities can be assessed to determine if any are abnormal at about 16 to 20 weeks gestation.

An ultrasound examination can be useful to determine the size and position of the fetus, the amount of amniotic fluid, and the appearance of fetal anatomy. Measurements carried out on ultrasound images may include one or more of the following ultrasound markers of a chromosomal variation: nuchal translucency (NT) thickness, nuchal fold thickness, femur length, humerus length, hyperechogenic bowel, renal pyelectasis, fetal heart rate, and certain cardiac abnormalities. In some embodiments, ultrasound is the first, second, third or a subsequent test in the prenatal screen/diagnosis disclosed herein.

### Nuchal Translucency Screening Test

A nuchal translucency (NT) screening test is a sonographic prenatal screening scan (ultrasound) to help identify higher risks of genetic conditions in a fetus (e.g., Down syndrome). The scan is carried out at 11-13.6 weeks pregnancy and assesses the amount of fluid behind the neck of the fetus. Fetuses at risk of Down syndrome tend to have a higher amount of fluid around the neck. The scan may also help confirm both the accuracy of the pregnancy dates and the fetal viability. Its high definition imaging may also detect other less common chromosomal abnormalities.

Nuchal scan is typically performed between 11 and 14 weeks of gestation. The scan is obtained with the fetus in sagittal section and a neutral position of the fetal head (neither hyperflexed nor extended, either of which can influence the nuchal translucency thickness). The fetal image is enlarged to fill 75% of the screen, and the maximum thickness is measured, from leading edge to leading edge. Normal thickness depends on the crown-rump length (CRL) of the fetus. Among those fetuses whose nuchal translucency exceeds the normal values, there is a relatively high risk of significant abnormality. Use of ultrasound and nuchal scans in combination with the methods and systems described herein is specifically contemplated. In some embodiments, nuchal scan is the first, second, third or a subsequent test in the prenatal screen/diagnosis disclosed herein.

### Detecting Fetal Genetic Variations

### Fetal Genetic Variation

As used herein, fetal genetic variation means any variation in a fetal nucleic acid sequence. Genetic variations can range from a single base pair variation to a chromosomal variation, or any other variation known in the art. Genetic variations can be simple sequence repeats, short tandem repeats, single nucleotide polymorphisms, translocations, inversions, deletions, duplications, or any other copy number variations. In some embodiments, the chromosomal variation is a chromosomal abnormality. For example, the chromosomal variation can be aneuploidy, inversion, translocation, a deletion, or a duplication. A genetic variation can also be mosaic. For example, the genetic variation can be associated with genetic conditions or risk factors for genetic conditions (e.g., cystic fibrosis, Tay-Sachs disease, Huntington disease, Alzheimer disease, and various cancers). Genetic variations can also include any mutation, chromosomal abnormality, or other variation disclosed in the documents incorporated herein by reference (e.g., aneuploidy, microdeletions, or microduplications). Genetic variations can have positive, negative, or neutral effects on phenotype. For example, chromosomal variations can include advantageous, deleterious, or neutral variations. In some embodiments, the fetal genetic variation is a risk factor for a disease or disorder. In some embodiments, the fetal genetic variation encodes a desired phenotypic trait.

Fetal DNA from Maternal Blood

Fetal DNA has been detected and quantitated in maternal plasma and serum (Lo et al., Lancet 350:485-487 (1997); Lo et al., Am. J. hum. Genet. 62:768-775 (1998)). Multiple fetal cell types occur in the maternal circulation, including fetal granulocytes, lymphocytes, nucleated red blood cells, and trophoblast cells (Pertl, and Bianchi, Obstetrics and Gynecology 98: 483-490 (2001)). Fetal DNA can be detected in the serum at the seventh week of gestation, and increases with the term of the pregnancy.

Circulating fetal DNA has been used to determine the sex of the fetus (Lo et al., Am. J. hum. Genet. 62:768-775 (1998)). Fetal rhesus D genotype has been detected using fetal DNA. Methods for detecting genetic conditions in cell-free fetal nucleic acids from biological samples are known and available to one of skill in the art. (See, e.g., Wright et al. (2009) Hum Reprod Update 15:139-51). For example, fetal aneuploidy can be detected from cell-free fetal DNA using digital PCR (U.S. Patent Application Publication Nos. 2007/0202525, 2009/0053719). This technique uses dilution to isolate single template DNA molecules to be amplified, in order to detect very small differences in chromosome ratios. This technique further provides a measure of the total (i.e., fetal plus maternal) dosage of a particular chromosome relative to another reference chromosome. Fetal aneuploidy can also be detected from cell-free fetal DNA using massively parallel genomic sequencing techniques (U.S. Patent Application Publication No. 2009/0029377). Direct shotgun sequencing has recently been utilized to detect fetal aneuploidy (U.S. Patent Application Publication No. 2010/0138165). Using simultaneous sequencing of millions of short DNA fragments, it is possible to compare the amount of sequences produced from different chromosomes to detect any small over-representation caused by aneuploidy, or potentially smaller chromosomal imbalances, in the fetus. Single molecule sequencing techniques may also be used to detect genetic variations in cell-free fetal DNA (U.S. Patent Application Publication No. 2010/0216153). Use of any one of these techniques is specifically contemplated in embodiments of the systems and methods provided herein. In some embodiments, one of the above techniques for detecting fetal genetic variations is the first, second, third or a subsequent test in the prenatal screen/diagnosis disclosed herein.

Fetal Cells Isolated from Maternal Blood

The presence of fetal nucleated cells in maternal blood makes it possible to use these cells for noninvasive prenatal diagnosis (Walknowska, et al., Lancet 1:1119-1122, 1969; Lo et al., Lancet 2:1363-65, 1989; Lo et al., Blood 88:4390-95, 1996). The fetal cells can be sorted and analyzed by a variety of techniques to look for particular DNA sequences (Bianchi et al., Am. J. Hum. Genet. 61:822-29, (1997); Bianchi et al., PNAS 93:705-08, (1996)). Fluorescence in-situ hybridization (FISH) is one technique that can be applied to identify particular chromosomes of the fetal cells recovered from maternal blood and diagnose aneuploid conditions such as trisomies and monosomy X. Also, it has been reported that the number of fetal cells in maternal blood increases in aneuploid pregnancies.

Methods for measuring and detecting fetal genetic variations from a fetal cells present in a biological sample have been described. (See, e.g., International Publication No. WO 2010/075459). In some embodiments, the detection of a fetal genetic variation in a cellular portion of the biological sample is achieved by obtaining a cellular portion of a biological sample comprising a mixture of maternal and fetal cells; dividing the sample into subsamples; screening or genotyping a subsample for the presence of a fetal genetic variation; and identifying the presence of at least one fetal genetic variation from a fetal cell in at least one subsample. Subsamples can be screened or genotyped for a fetal genetic variation using a number of methods known in the art, including those disclosed in International Publication No. WO 2010/075459. For example, subsamples can be screened or genotyped using molecular beacons or other nucleic acid-based SNP detection methods. In some embodiments, the nucleic acid template in a subsample is amplified and detected (e.g., using PCR-based methods). However, in some embodiments, the nucleic acid template is not amplified. In some embodiments, one of the above techniques for detecting fetal genetic variations is the first, second, third or a subsequent test in the prenatal screen/diagnosis disclosed herein.

### Assays in Fetal DNA and Fetal Cells

### Enrich for Fetal Cells

In some embodiments of the invention, a mixed sample of maternal and fetal cells is enriched for fetal cells prior to performing the methods described herein. For example, in some embodiments, fetal cells in a mixture of maternal and fetal cells have been enriched to about 1 in 10, about 1 in 100, about 1 in 1000, about 1 in 10000, or about 1 in 100000 fetal:maternal cells, or a range defined by any two of the preceding values. Samples can be enriched for fetal cells through positive selection, negative selection, or a combination of positive and negative selection. In some embodiments, fetal cells are directly captured. In other embodiments, maternal cells are captured and fetal cells are collected from the remaining sample.

Samples can be enriched for fetal cells based on differences in the physical properties of cells. For example, samples can be enriched for fetal cells based on density, cell membrane structure, or morphology. In some of the embodiments based on density, density gradients such as FICOLL™ (GE Healthcare Life Sciences, Piscataway, N.J.), PERCOLL™ (GE Healthcare Life Sciences, Piscataway, N.J.), iodixanol (Axis Shield, Oslo, Norway), NYCODENZ® (Axis Shield, Oslo, Norway), or sucrose are used. In some of the embodiments based on cell membrane structure, a lysis reagent (e.g., ammonium chloride) is used. In some of the embodiments based on morphology, flow cytometry or filters are used. Samples can also be enriched for fetal cells based on other physical properties known in the art. For example, samples can be enriched for fetal cells based on dielectric or magnetic properties. Further, samples can be enriched for fetal cells by collecting bone marrow.

Samples can also be enriched for fetal cells based on differences in the biochemical properties of cells. For example, samples can be enriched for fetal cells based on antigen, nucleic acid, metabolic, gene expression, or epigenetic differences. In some of the embodiments based on antigen differences, antibody-conjugated magnetic or paramagnetic beads in magnetic field gradients or fluorescently labeled antibodies with flow cytometry are used. In some of the embodiments based on nucleic acid differences, flow cytometry is used. In some of the embodiments based on metabolic differences, dye uptake/exclusion measured by flow cytometry or another sorting technology is used. In some of the embodiments based on gene expression, cell culture with cytokines is used. In some of the embodiments based on epigenetic differences, cell culture is used. Samples can also be enriched for fetal cells based on other biochemical properties known in the art. For example, samples can be enriched for fetal cells based on pH or motility. Further, in some embodiments, more than one method is used to enrich for fetal cells.

In some embodiments, samples are enriched for fetal cells by removing red blood cells through the use of lysis reagents such as ammonium chloride or by separation using density gradients such as FICOLL™ (Sigma-Aldrich, St. Louis, Mo.), PERCOLL™ (GE Healthcare Life Sciences Piscataway, N.J.), or sucrose. A density gradient can also be used to reduce the white cell fraction. The resulting peripheral blood mononuclear cells (“PBMCs”) can be further enriched for fetal cells using magnetic bead separation techniques from manufactures such as Miltenyi Biotec (Gladbach, Germany), Stemcell Technologies (Vancouver, BC, Canada), and Dynal Biotech/Invitrogen (Carlsbad, Calif.). Positive enrichment, negative depletion, or a combination of both can be used to enrich the fetal fraction in the PBMCs.

While no fetal-specific surface markers are currently known, there are several markers that have been shown to positively enrich fetal cells to 1 fetal cell in 1,000 to 100,000 maternal cells. In some embodiments, CD71, CD34, CD45, or CD235a cell surface markers are used to enrich fetal cells. In some embodiments, cell surface markers that are not found on fetal cell populations are used to negatively enrich fetal cells by depleting adult cell populations. In some embodiments, combinations of CD2, CD3, CD11b, CD14, CD15, CD16, CD19, CD45, CD56, CD123 and CD61 are used to deplete adult cells. Flow cytometry sorting may also be used to further enrich for fetal cells using cell surface markers or intracellular markers conjugated to fluorescent labels. Intracellular markers may include nuclear stains or antibodies against intracellular proteins preferentially expressed in fetal cells (e.g., fetal hemoglobin).

Oxidation of hemoglobin has been identified as one way to preferentially enrich nucleated red blood cells (NRBCs) using magnetic field gradients (Zborowski, Biophys J 2003 84(4): 2638-2645). In addition, microfluidic devices have been developed which facilitate separation of red cells from white cells or enrich fetal cells from PBMCs (Huang, Prenatal Diagnosis 2008 28: 892-899).

In some embodiments, samples are enriched for fetal cells by differentially expanding fetal cells over maternal cells in culture. Differential expansion can be performed by any number of methods known in the art, including incubating cells from a sample of maternal blood containing CD34+ cells of both maternal and fetal origin in the presence of Stem Cell Factor (SCF) in serum free media as described in WO 2008/048931, which is herein incorporated by reference in its entirety

In some embodiments of the invention, fetal cells in a mixture of maternal and fetal cells are enriched to about 1 in 2, about 1 in 5, about 1 in 10, about 1 in 100, about 1 in 1000, about 1 in 10000, or about 1 in 100000 fetal:maternal cells, or a range defined by any two of the preceding values.

Divide into Subsamples

Prior to the screening or genotyping described herein, samples can be divided into subsamples with few enough cells such that the chromosome copy number from the samples is preserved in the subsamples, even following amplification (e.g., whole genome amplification (WGA) or whole transcriptome amplification (WTA)). Samples can be divided into subsamples consistent with a Poisson Distribution or a non-Poisson Distribution. In some embodiments, samples are divided sequentially. For example, samples can be divided in serial. In other embodiments, samples are divided in parallel.

In some embodiments, samples are divided to provide subsample volumes of, less than, or less than about, 100 uL, 50 uL, 10 uL, 1000 nL, 500 nL, 400 nL, 300 nL, 200 nL, 100 nL, 50 nL, 30 nL, 10 nL, 3 nL, or 1 nL, or a range defined by any two of the preceding values. Preferably, each subsample contains a volume not more than 100 nL. In some embodiments, each subsample comprises not more than about 500, 400, 300, 200, or 100 cells, or a range defined by any two of the preceding values. Preferably, each subsample comprises not more than about 50, 40, 30, 20, or 10 cells, or a range defined by any two of the preceding values. More preferably, each subsample comprises not more than about 5, 4, 3, or 2 cells. In some embodiments, each subsample comprises not more than one cell. In some embodiments, each subsample comprises one or zero cells.

In some embodiments, each subsample comprises an average of, or of about, 500, 400, 300, 200, or 100 cells, or a range defined by any two of the preceding values. Preferably, each subsample comprises an average of about 50, 40, 30, 20, or 10 cells, or a range defined by any two of the preceding values. More preferably, each subsample comprises an average of, or of about, 5, 4, 3, or 2 cells, or a range defined by any two of the preceding values. In some embodiments, each subsample comprises an average of less than about one cell, about one cell, or about one to two cells, or a range defined by any two of the preceding values.

The division of samples is performed by any method known in the art, including the use of oil plugs to create oil separation of individual cells in a microfluidic device, deposition into wells, or free-standing drops anchored by surface tension to a flat substrate. In addition, a subsample can be suspended in a buffer that will be appropriate for subsequent reactions. For example, a subsample can suspended in a solution comprising lysis and PCR buffers that will allow for a single-step cell lysis followed by amplification without further manipulation of subsamples.

### Amplify Subsamples

To compensate for the limited amount of genetic material in a single cell or subsample, subsample amplification can optionally be performed. For example, nucleic acid replication or cell division can be performed. Samples can be divided into subsamples with few enough cells such that the chromosome copy number from the samples is preserved in the subsamples following subsample amplification. In a preferred embodiment, subsample amplification is performed on a subsample containing a single cell, so that the resulting amplified product represents the genome or transcriptome of either a maternal or fetal cell. For example, subsample amplification can be performed on individual cells that are located in microwells or in drops separated by oil plugs as described herein.

Nucleic acid replication can be performed using any method for generating additional copies of nucleic acids, additional signals indicative of nucleic acids, or other proxies for nucleic acids (e.g., protein expression) known in the art. In some embodiments, nucleic acid replication is performed using WGA, WTA, or targeted nucleic acid amplification techniques. In other embodiments, nucleic acid replication is performed using methods that generate a signal indicative of nucleic acid sequences, such as INVADER® (Hologic, Inc., Bedford, Mass.). In some embodiments, only a portion of the amplified sequence is complementary to the nucleic acid template. For example, in some embodiments, a contiguous amplified product contains a portion of the nucleic acid template and a portion of a signal sequence. General techniques for nucleic acid replication can include isothermal or thermocycled replication. For example, in some embodiments, isothermal WGA is performed. In some embodiments, at least a subset of samples undergo more than one WGA reaction. In some embodiments, nucleic acid replication is performed prior to SNP genotyping. However, nucleic acid replication can also be performed after SNP genotyping.

Cell replication can also be performed using any method known in the art. In some embodiments, cells are cultured in media and supplements to generate additional nucleic acid copies for use in the methods described herein. In other embodiments, cells are cultured and one or more cell is left intact for use in subsequent analysis. In some embodiments, cell replication is performed prior to division into subsamples. Preferably, cell replication is performed after division into subsamples.

Divide into Aliquots

Following subsample amplification, amplified products can be divided into aliquots. These aliquots can be used for a plurality of assays. For example, in one embodiment, one or more of the aliquots from an amplified product is used to detect the presence of a fetal allele, while one or more of the other aliquots is used to detect the presence of a fetal genetic variation in an amplified product that contains a fetal genome or transcriptome. In some embodiments, an aliquot identified as containing a fetal genome or transcriptome is assayed by array for genetic variations. For example, an aliquot can be assayed for genetic variations associated with genetic conditions (e.g., Williams syndrome, Wolf-Hirschhorn syndrome, Miller-Dieker syndrome, Smith Magenis syndrome, Angelman syndrome, Di George syndrome, Prader-Willi syndrome, Jacobsen syndrome, Cri du chat syndrome, Charcot-Marie-Tooth disease, microduplication 22q11.2 syndrome, cystic fibrosis, Tay-Sachs disease, Huntington disease, Alzheimer disease, and various cancers).

Homogenous or non-homogeneous portions of amplified products can be selected for division into aliquots. In some embodiments, homogenous portions of amplified products are divided sequentially (e.g., in serial). In other embodiments, homogeneous portions of amplified products are divided in parallel. Alternatively, non-homogeneous portions of amplified products can be selected for division into aliquots using positive selection, negative selection, or a combination of positive and negative selection. For example, in some embodiments, bead-bound capture oligos are used to target desired portions of amplified products for division into aliquots. In other embodiments, surface-bound oligos are used to eliminate undesired portions of amplified products. Non-homogeneous portions of amplified products can be selected based on any physical or biochemical property known in the art, including those described herein. For example, portions of amplified products with a particular charge, size, or chromosomal identity can be selected for division into aliquots.

In some embodiments where the optional step of subsample amplification is not carried out, a portion or aliquot of a subsample can be removed for subsequent analysis.

In some embodiments, aliquots are pooled into groups of two or more aliquots. This allows the number of SNP-based or other reactions described herein to be reduced by as much as a factor of N, where N is the number of aliquots in each pool. Aliquots from positive pools (i.e., pools with at least one genotype differing from the maternal genotype) may then be retested aliquot-by-aliquot to identify the aliquot containing a fetal allele. In some embodiments, each pool is tested for a non-maternal allele at a test locus. In some embodiments, each pool is tested for non-maternal alleles at two or more test loci.

In some embodiments, aliquots are pooled using an indexing system that allows for identification of the source of a positive aliquot within a positive pool. For example, two or more aliquots may be taken from each amplified product to form indexed pools of N×M amplified aliquots. Wells containing at least one fetal allele can be identified by locating the intersection of positive N and M pools in the orthogonal ordinate system. In some embodiments, N (i.e., the number of columns in the N×M index) and M (i.e., the number of rows in the N×M index) are independently between about 2 and about 1000. Preferably, N and M are independently between about 8 and about 100. In some embodiments, N is, is about, is at least, is at least about, is not more than, is not more than about 2, 3, 4, 5, 6, 7, 8, 9, 10, 11, 12, 13, 14, 15, 16, 17, 18, 19, 20, 21, 22, 23, 24, 30, 32, 36, 40, 48, 50, 56, 60, 64, 70, 72, 80, 84, 88, 90, 96, 100, 192, 288, 384, 480, 576, 672, 768, 864, 960, 1000, or a range defined by any two of the preceding values. In some embodiments, M is, is about, is at least, is at least about, is not more than, is not more than about 2, 3, 4, 5, 6, 7, 8, 9, 10, 11, 12, 13, 14, 15, 16, 17, 18, 19, 20, 21, 22, 23, 24, 30, 32, 36, 40, 48, 50, 56, 60, 64, 70, 72, 80, 84, 88, 90, 96, 100, 192, 288, 384, 480, 576, 672, 768, 864, 960, 1000, or a range defined by any two of the preceding values. In preferred embodiments, homogeneous or non-homogeneous portions of amplified products are indexed to allow for identification of the source of positive aliquots.

### Obtain or Infer a Parental Genotype

Parental genotypes can be obtained or inferred to aid in the identification of non-maternal alleles. Information regarding non-maternal alleles can in turn be used to screen or genotype aliquots or subsamples, or to directly analyze fetal genomes for genetic variants. For example, paternal or maternal genotypes can be obtained by directly genotyping paternal or maternal samples, or inferred by genotyping samples from genetically related family members. However, in a preferred embodiment, there is no need to obtain or infer a paternal genotype. For example, in a preferred embodiment, only a maternal genotype is obtained.

In some embodiments, paternal or maternal genotypes are obtained by genotyping genetic material from blood, plasma, serum, urine, buccal swab, saliva, tears, skin, or any other source of paternal nucleic acids (including those described herein). In some embodiments, paternal or maternal genotypes are obtained using cell-free nucleic acids or nucleic acids extracted from cells derived from one of these sources. Paternal or maternal genotyping is also preferably performed on DNA, but can also be performed on RNA, cDNA, or any other nucleic acid known in the art. In some embodiments, the template of a paternal or maternal sample is amplified and detected (e.g., using PCR-based methods). However, in some embodiments, the template of a paternal or maternal sample is not amplified (e.g., using the methods described herein).

Paternal and maternal genotypes can also be obtained by accessing information generated during prior genetic testing, such as information in a database, in a test report, or from a previous pregnancy for which a method described herein was performed. Paternal or maternal genotypes can also be inferred using the genotypes of blood relatives. For example, the genotypes of genetically related parents, siblings, grandparents, aunts, uncles, or children can be used to infer a paternal or maternal genotype.

Any polymorphism known in the art can be used to genotype a parental sample. For example, SNPs, haplotypes, short tandem repeats (STRs), or other sequence variations can be genotyped. Other genetic or epigenetic markers can also be used to genotype a parental sample. For example, copy number variations (CNVs) or methylation patterns can be assessed.

### Optionally Identify at Least One Non-Maternal Allele in a Mixture of Maternal and Fetal Nucleic Acids

In embodiments where a homozygous target locus has been identified in a maternal sample, a mixture of maternal and fetal nucleic acids can optionally be used to identify a heterozygous genotype at the same locus, which indicates the presence of a fetal (i.e., non-maternal allele/informative paternal allele). This optional step is preferably performed prior to screening or genotyping the individual aliquots or subsamples. By screening for a non-maternal allele, and thereby identifying a heterozygous locus (and therefore a fetal allele) in the mixed sample of maternal and fetal nucleic acids, the aliquots and subsamples can be more efficiently screened for SNPs that are known to be informative. In a preferred embodiment, fetal cell-free DNA is used to screen for the non-maternal allele and identify the heterozygous locus. In another embodiment, fetal cell-free RNA or cDNA is used to identify the heterozygous locus.

Cell-free nucleic acids can be obtained from any source known in the art, including blood, serum, plasma, urine, cervical swab, cervical lavage, uterine lavage, or culdocentesis from a pregnant woman. Nucleic acids can also be extracted from a mixed sample of maternal and fetal cells to identify the heterozygous locus. Preferably, DNA is extracted from a mixed sample of maternal and fetal cells. However, RNA or cDNA can be extracted from a mixed sample of maternal and fetal cells. Nucleic acids can be extracted from cells obtained from any source known in the art, including blood, cervical swab, cervical lavage, uterine lavage, culdocentesis, lymph node, or bone marrow. In other embodiments, whole blood is used to identify a heterozygous genotype without (or prior to) dividing the whole blood into a cellular, plasma, or serum fraction.

In some embodiments, the nucleic acid template of an aliquot from a mixed sample of maternal and fetal nucleic acids is amplified and detected to identify a heterozygous locus (e.g., using PCR-based methods). However, in some embodiments, the nucleic acid template of an aliquot from a mixed sample is not amplified to identify a heterozygous locus. For example, methods in which only a signal associated with the template is amplified (e.g., the ABSCRIPTION™ method as described in U.S. Pat. Nos. 7,226,798, 7,473,775, and 7,468,261 (Ribomed Biotechnologies, Inc., Carlsbad, Calif.) or methods involving the INVADER® chemistry (Hologic, Inc.)) can be employed. Methods in which the nucleic acid template is detected without amplification of the signal or the template (e.g., the method involving chemical detection of DNA binding as described in WO 2005/01122 (Adnavance Technologies, Inc., San Diego, Calif.)) can also be employed. In addition, methods in which the nucleic acid template is sequenced without amplification (e.g., the sequencing method as described in Eid et al., Science 2009 323(5910): 133-38) can be employed. It will be understood by one of skill in the art that the source of the template for the identification of a non-maternal allele can include serum, plasma, urine, a cervical swab, or any other source of nucleic acids known in the art.

### Screen or Genotype at Least One Aliquot or Subsample for the Presence of at Least One Informative Paternal Allele

The next steps are to screen or genotype subsamples or aliquots to identify non-maternal alleles. Once a SNP panel has been generated (as described in more detail herein) and a set of target loci for which the maternal genetic sample is homozygous (or, alternatively, heterozygous) has been identified, a test locus or loci can be selected to screen or genotype the subsample or aliquot for the presence of a fetal allele. To detect the presence of a fetal allele, a test locus is screened or genotyped for the presence of a non-maternal allele (i.e., by identifying a heterozygous, or alternatively homozygous genotype at the test locus). In some embodiments, aliquots of an amplified product are screened or genotyped aliquot-by-aliquot to detect a heterozygous (or, alternatively, homozygous) locus. In some embodiments, an aliquot contains amplified material from a single cell.

A subsample or aliquot can be genotyped at a locus previously identified as homozygous in a maternal sample. In some embodiments, the genotype at a locus previously identified as homozygous in a maternal sample is determined by screening for the presence of a non-maternal allele. Optionally, as described herein, prior to screening the aliquots or subsample for the non-maternal allele, a sample of mixed maternal and fetal nucleic acids (preferably cell-free) can be used to identify the presence of a non-maternal allele in the mixed nucleic acid, indicating the presence of a heterozygous genotype at the same locus in the fetal material.

In other embodiments, a subsample or aliquot is genotyped at a locus previously identified as heterozygous in a maternal sample. The identification of a homozygous genotype in the aliquot or subsample for the same locus indicates the presence of a non-maternal, i.e., fetal allele.

It will be understood by one of skill in the art that the screening or genotyping methods in any of the embodiments described herein may be performed using either aliquots or subsamples. Aliquots and subsamples can be screened or genotyped for a fetal allele using a number of methods known in the art, including those mentioned herein. For example, aliquots can be screened or genotyped using molecular beacons or other nucleic acid-based SNP detection methods. In some embodiments, the nucleic acid template in an aliquot is amplified and detected (e.g., using PCR-based methods). However, in some embodiments, the nucleic acid template is not amplified (e.g., using the methods described herein). Further, any marker known in the art can be used to screen or genotype aliquots and subsamples. In preferred embodiments, the SNP, haplotype, short tandem repeat (STR), other sequence variation, copy number variation (CNV), or epigenetic marker genotyped in the maternal sample is used.

Because in some embodiments subsamples comprise not more than one cell, the screening or genotyping of a subsample or aliquot can be cell-by-cell. Subsamples or aliquots can also be screened or genotyped using a number of methods known in the art, including those mentioned herein. Preferably, subsamples are screened for heterozygous alleles using quantitative PCR (qPCR) with a TAQMAN® system (Foster City, Calif.). A predetermined number of subsamples or cells can be screened to detect a heterozygous allele. For example, as shown in Table 1, to test 7 loci in a sample enriched to 1:10,000 fetal:maternal cells, where approximately 5 fetal cells per loci are expected, the predetermined number of samples or cells is 350,000. In some embodiments, the number of fetal cells per loci is, is about, is at least, is at least about, is not more than, is not more than about 1, 2, 3, 4, 5, 6, 7, 8, 9, 10, 15, 20, 25, 30, 35, 40, 50, 60, 70, 80, 90, 100, 125, 150, 175, 200, 225, 250, 275, or 300, or a range defined by any two of the preceding values.

TABLE 1

| | | Total Enriched Cells (in Thousands) | | | | | | | |
|---|---|---|---|---|---|---|---|---|---|
| | | 50 | 100 | 150 | 200 | 250 | 300 | 350 | 400 |
| | | Number of loci possible with 5 fetal cells per loci | | | | | | | |
| Fetal Frequency | 1:100 | 100 | 200 | 300 | 400 | 500 | 600 | 700 | 800 |
| | 1:1,000 | 10 | 20 | 30 | 40 | 50 | 60 | 70 | 80 |
| | 1:10,000 | 1 | 2 | 3 | 4 | 5 | 6 | 7 | 8 |

### Identify at Least One Informative Paternal Allele in at Least One Aliquot or Subsample

As described herein, after homozygous (or, alternatively, heterozygous) SNPs are identified in maternal genetic material, these SNPs can be screened or genotyped in the subsamples or aliquots to detect the presence of a non-maternal allele. If a first maternal homozygous/heterozygous SNP does not generate a heterozygous/homozygous genotype in the aliquots or subsamples, a second maternal homozygous/heterozygous SNP can be selected and genotyped. This process can be repeated until a non-maternal allele is detected or until a predetermined number of SNPs and/or cells, subsamples or aliquots are screened. The process can also involve genotyping multiple aliquots or subsamples, multiplexing SNPs, or any combination thereof.

Test loci are screened or genotyped such that at least one fetal allele will be detected if present in a sample. In some embodiments, test loci are screened or genotyped until at least one non-maternal allele is detected. In some embodiments, a first test locus is screened or genotyped in aliquots or subsamples until enough cells are genotyped to exceed a designated probability of detecting a non-maternal allele. If a non-maternal allele is not detected, a second test locus is screened or genotyped in aliquots or subsamples until enough cells are run to exceed a designated probability of detecting a non-maternal allele. If a non-maternal allele is not detected, additional test loci are screened or genotyped until the number of test loci run exceeds a designated cumulative probability of detecting a non-maternal (and therefore fetal) allele from a mixed sample.

Test loci can also be screened or genotyped until more than one fetal allele is detected. If more than one test locus is screened or genotyped, each additional locus testing positive for a fetal allele (i.e., with a genotype differing from the maternal genotype) increases the confidence of detecting a fetal genome in a subsample or aliquot. In addition, a predetermined number of test loci can be designated to account for the fact that fetal genetic material may not be present. In some embodiments, a predetermined number of test loci is determined by calculating the cumulative probability of detecting a fetal allele for a relevant set of variables. For example, as shown in Table 3, the probability of detecting the presence of a heterozygous fetal allele using 7 SNPs with a minor allele frequency of about 0.5 is 1−(0.5)(0.5)(0.5)(0.5)(0.5)(0.5)(0.5)=99.22%. As shown in Table 1, for 350,000 aliquots or subsamples from a sample enriched for 1:10,000 fetal:maternal cells, the predetermined number of test loci can therefore be about 7 test loci.

Test loci can be screened or genotyped individually or in multiples. In some embodiments, one test locus is screened or genotyped in a single aliquot or subsample. A single test locus can also be screened or genotyped in multiple aliquots or subsamples. In addition, aliquots or subsamples can be screened or genotyped individually or in multiples. In some embodiments, more than one test locus is screened or genotyped in a single aliquot or subsample. In addition, more than one test locus can also be screened or genotyped in multiple aliquots or subsamples. In some embodiments, the number of test loci in a panel is, is about, is at least, is at least about, is not more than, is not more than about 2, 3, 4, 5, 6, 7, 8, 9, or 10 test loci multiplexed to screen for or genotype a fetal allele, or a range defined by any two of the preceding values.

In some embodiments, a locus identified as containing a heterozygous (and therefore non-maternal) genotype in a mixture of maternal and fetal nucleic acids (preferably cell-free) is screened subsample-by-subsample with subsamples derived from a mixture of maternal and fetal cells. Conserved aliquots and subsamples can then be used to perform additional genetic analyses. However, despite the advantages of maternal and fetal cell-free nucleic acids in detecting the presence of a fetal allele, these samples are not suitable for analyses that require preservation of the integrity of the fetal genome or transcriptome, or capture of samples. This highlights one of the benefits of using the cell-based methods described herein.

### Collect Aliquots or Subsamples

Aliquots or subsamples identified as containing a fetal allele can be collected for subsequent analyses. The aliquot(s) collected for subsequent analysis can be the same one(s) used to screen for the fetal allele, or a different aliquot from the same subsample can be used to provide an aliquot that has not be subject to any reactions used for fetal allele screening. In some embodiments, aliquots or subsamples are selected for collection based on quality, quantity, or the presence of desired nucleic acids. For example, aliquots or subsamples can be selected for collection using signal correlations, signal intensities, signal intensity ratios, signals compared to a background measurement, assay kinetics plotted against time, assay kinetics plotted against temperature, or other performance metrics known in the art. In some embodiments, the marker or region used to discriminate paternal from maternal alleles is collected. However, in other embodiments, an unlinked marker or region is collected for further analysis.

In some embodiments, only a desired portion of an aliquot or subsample is collected for subsequent analysis. For example, in some embodiments, a hybridization probe is used to collect only nucleic acid sequences from a chromosome or region of interest. In other embodiments, an entire aliquot, subsample, or a homogenous portion thereof is collected.

### Analyze at Least One Fetal Genome for Genetic Variants

Aliquots or subsamples identified as having a non-maternal allele, optionally collected as described herein, can be further analyzed, for example, to test for the presence of a chromosomal or genetic variation. The aliquot used for analysis can be the same one screened or genotyped for the fetal allele. Or the aliquot used for analysis can be a different aliquot from the same subsample, but one that was not subject to any screening or genotyping for a fetal allele. Entire fetal genomes or portions thereof can be selected for further analyses. In some embodiments, polymorphisms are genotyped using methods known in the art. For example, SNPs, haplotypes, or STRs can be genotyped using PCR and, if appropriate, subsequent detection methods such as capillary electrophoresis. Polymorphisms can also be genotyped using sequencing methods. Genotyping is preferably performed using high throughput techniques. For example, in some embodiments, a microarray is used to generate data regarding SNPs and/or haplotypes. Copy number variation can also be assessed for further analyses. For example, array comparative genomic hybridization (aCGH) can be used to detect copy number variations. Chromosomal rearrangements can also be assessed. For example, inversions or translocations can be detected using methods such as sequencing, FISH, or PCR.

In some embodiments, a ratio of maternally- and paternally-inherited alleles is determined to analyze the presence of a genetic variation. Optionally, the same locus is used to determine the presence of a fetal allele and the presence of a genetic variation. For example, intensity of the alleles at a heterozygous test locus can be measured, with a 2:1 or 1:2 intensity ratio indicating copy number variation. However, in some embodiments, the locus used to determine the presence of a fetal allele is not the same locus used to determine a genetic variation. In addition, it will be understood by one of skill in the art in any of the embodiments described herein that other intensity ratios (e.g., 3:1, 1:3, 3:2, 2:3, 4:1, and 1:4) can be used to detect the presence of copy number variation.

In some embodiments, an overrepresentation or underrepresentation of chromosomal sequences is determined to analyze the presence of copy number variation. For example, the number of unique sequence reads for a particular chromosome can be measured and compared to a maternal and/or other reference chromosome, with a ratio less than or greater than 1:1 indicating a copy number variation. The detection of these unique sequence reads can be performed using small scale (e.g., sequencing with primer pairs designed for specific loci) or large scale (e.g., sequencing of the entire genome) methods. The number of sequence reads for a particular chromosomal region can also be measured and compared to a maternal and/or other reference chromosomal region, with a ratio less than or greater than 1:1 indicating the presence of a copy number variation.

Aliquots or subsamples may be analyzed individually or in a combined sample of at least two aliquots or subsamples. In some embodiments, aliquots or subsamples are ranked based on signal metrics as described herein and a preferred set is selected for analysis or pooling followed by analysis. In some embodiments, isolated aliquots or subsamples or pools are tested for the presence of a genetic or chromosomal variation using array comparative genomic hybridization (aCGH), quantitative fluorescence PCR (QF-PCR), short tandem repeat (STR) analysis, or sequencing. However, any technique known in the art, including those described herein, can be used to test for the presence of a genetic or chromosomal variation.

Screening or genotyping aliquots or subsamples on a cell-by-cell basis allows for the detection of mosaicism (i.e., a condition in which cells from the same individual have different genetic profiles). For example, subsamples can be screened or genotyped to detect a mosaic chromosomal variation. In some embodiments, the number of subsamples screened or genotyped for mosaicism is about 2 to about 100 subsamples. In some embodiments, the number of subsamples screened or genotyped is, is about, is at least, is at least about, is not more than, is not more than about 2, 3, 4, 5, 6, 7, 8, 9, 10, 15, 20, 25, 30, 35, 40, 50, 60, 70, 80, 90, or 100, or a range defined by any two of the preceding values. In a preferred embodiment, the number of subsamples screened or genotyped is about 5 to about 10 subsamples. Optionally, the same locus is used to determine the presence of a fetal allele and the presence of mosaicism. For example, intensity of the alleles at a heterozygous test locus can be measured, with a 1:1 intensity ratio in at least one subsample and a 2:1 or 1:2 intensity ratio in at least one other subsample indicating the presence of a mosaic genetic variation. However, different loci can also be used to determine the presence of a fetal allele and the presence of mosaicism. For example, a homozygous test locus can be used to identify a fetal allele. A heterozygous locus can then be detected and the intensity of the alleles at the heterozygous locus can be measured, with a 1:1 intensity ratio in at least one subsample and a 2:1 or 1:2 intensity ratio in at least one other subsample again indicating the presence of a mosaic genetic variation.

In some embodiments, mosaicism is detected using a sex-specific chromosome. For example, alleles at a heterozygous X chromosome test locus can be detected, with the presence of one allele in at least one subsample and the presence of both alleles in at least one other subsample indicating the presence of mosaic aneuploidy (e.g., mosaic Turner syndrome). In another example, the presence of alleles at a homozygous X chromosome test locus can be detected, with a 1:1 X:Y chromosome intensity ratio in at least one subsample and a 2:1 X:Y chromosome intensity ratio in at least one other subsample indicating the presence of a mosaic aneuploidy (e.g., mosaic Klinefelter syndrome).

Screening or genotyping aliquots or subsamples on a cell-by-cell basis also allows for the detection of dizygotic twins (i.e., non-identical twins). For example, SNP genotyping can be performed on subsamples containing a fetal allele, with the presence of at least two subsamples with different SNP genotypes indicating the presence of dizygotic twins. In some embodiments, the number of SNPs screened or genotyped to detect dizygotic twins is about 1 to about 20 SNPs. In some embodiments, the number of SNPs screened or genotyped is, is about, is at least, is at least about, is not more than, is not more than about 1, 2, 3, 4, 5, 6, 7, 8, 9, 10, 11, 12, 13, 14, 15, 16, 17, 18, 19, or 20 SNPs, or a range defined by any two of the preceding values. In a preferred embodiment, the number of SNPs screened or genotyped is about 3 to about 4 SNPs. The probability of detecting the presence of dizygotic twins using 3 SNPs is about 1−(0.5)(0.5)(0.5)=87.5%, while the probability of detecting the presence of dizygotic twins using 4 SNPs is about 1−(0.5)(0.5)(0.5)(0.5)=93.75%, respectively.

In some embodiments, aliquots containing fetal alleles from dizygotic twins can be pooled. For example, aliquots comprising cells from a first twin can be pooled independently of aliquots comprising cells from a second twin. In some embodiments, pooled aliquots from a first and second twin can be independently placed on one or more arrays and assessed for genetic or chromosomal variation as described herein.

### Detection of Genetic Material in Low Abundance

Even if fetal DNA or RNA is present in a minor fraction of a sample comprising maternal and fetal genetic material, it is still possible to detect fetal SNP alleles using standard SNP detection formats, such as TAQMAN® PCR. However, to detect minor alleles, it may be necessary to optimize fluorescence detection to prevent maternal signal overlap from obscuring a fetal-specific signal. In some embodiments, optimal fluorescence dyes for probe labeling are selected to minimize overlap. For example, dyes separated across the standard fluorescence spectrum (400-700 nm) with less than 10% overlap between emissions can be selected. In this way, even minor signals (less than 10% signal intensity) from fetal alleles are not obscured by maternal signal (90% signal intensity). In some embodiments, optimal fluorescence filters are chosen to minimize overlap in emission detection and custom fluorescence detection hardware and software are chosen to minimize signal crosstalk. In some embodiments, digital PCR is used to optimize signal to background ratios.

Other potential tools for measuring allelic intensity include S-curve fitting and other statistical analyses known in the art. In some embodiments, Ct shift is measured to detect a genetic or chromosomal variation. For example, encapsulation of single cells for detection of fetal SNPs with TAQMAN® chemistry can allow for simultaneous detection of abnormal copy number for the SNP detected, and therefore the copy number of the corresponding chromosome. If a SNP resides on chromosome 21, the SNP abundance will correlate with a Ct value, and therefore a copy number for Chromosome 21. If a reaction contains a single cell, then the copy number of a chromosome in that cell can be detected by Ct shift. In this example, the use of the abundance of normal maternal cell Ct values establishes the Ct for a normal copy number of two chromosomes per cell and a Ct shift to an earlier cycle would indicate the presence of a copy number variation.

As described herein, sequencing methods can be used to screen for fetal alleles and/or to determine the presence of a genetic or chromosomal variation. In some embodiments, shotgun sequencing may be used as an alternative to CGH arrays to detect copy number variations (e.g., resulting from a genetic variation) as described, for example, in Xie and Tammi, BMC Bioinformatics 2009, 10(80). In some embodiments, whole genome sequencing may be performed.

SNP genotyping can also be performed using any method known in the art, including qPCR and TAQMAN® methods. A variety of SNP chemistries and platforms are available from manufacturers such as Life Technologies (TAQMAN®) (Carlsbad, Calif.), Illumina (GOLDENGATE®) (San Diego, Calif.), Millipore (AMPLIFLUOR®) (Billerica, Mass.), and DxS Ltd. (SCORPIONS™) (Manchester UK). Miniaturized formats are also available from BioTrove (OPENARRAY™) (Woburn, Mass.) and Fluidigm (BIOMARK™) (South San Francisco, Calif.).

### SNP Panels

A SNP panel can be used to identify target loci in a maternal genetic sample. Once these target loci are identified, they are used to identify the presence of a non-maternal allele in a mixed sample. Because genotyping a maternal genetic sample to identify a target locus is expensive and time consuming, a SNP panel is designed to include as few SNPs as possible. However, the panel must still include enough SNPs to identify a large enough set of target loci to allow for the detection of a fetal allele in a mixed sample, with these SNPs being sufficiently informative to conserve the finite quantity of cells in a mixture of fetal and maternal cells.

The size of a SNP panel is inversely related to the minor allele frequencies of the SNPs in the panel. In some embodiments of the invention, the goal is to identify about 1 to about 5 fetal cells from a mixed sample of maternal and fetal cells. The number of SNPs that must be assessed to achieve this goal depends on the minor allele frequency of SNPs that are assessed and the number of cells that are genotyped. The number of cells that must be assessed, in turn, depends on the extent of enrichment of the mixed sample for fetal cells.

In some embodiments, a SNP panel is therefore designed to minimize the number of tests necessary to identify loci which are homozygous (or, alternatively, heterozygous) in a maternal sample and heterozygous (or, alternatively, homozygous) in a mixed sample of maternal and fetal genetic material. In some embodiments, a SNP panel is designed to identify about 1 to about 20 homozygous maternal SNPs per chromosome-specific SNP panel. In some embodiments, the SNP panel is designed to detect a number of homozygous maternal SNPs per chromosome-specific panel that is, is about, is at least, is at least about, is not more than, is not more than about 1, 2, 3, 4, 5, 6, 7, 8, 9, 10, 11, 12, 13, 14, 15, 16, 17, 18, 19, or 20 SNPs, or a range defined by any two of the preceding values. In a preferred embodiment, a SNP panel is designed to identify about 5 to about 10 homozygous maternal SNPs per chromosome-specific SNP panel. More preferably, a SNP panel is designed to identify about 7 homozygous maternal SNPs per chromosome-specific SNP panel. For example, as shown in Table 2, for SNPs in HWE where p²=0.25, a panel of 20 SNPs is needed to identify 10 SNPs for which the maternal sample is homozygous.

Several chromosome-specific SNP panels, preferably comprising at least one control chromosome-specific panel, can be combined to create a SNP panel for genotyping maternal genetic material. Each chromosome-specific panel is designed to generate a target set of loci for that chromosome. Preferably, a chromosome-specific SNP panel comprises about 5 to about 100 unique SNPs. Preferably, the total number of SNPs in a chromosome-specific panel is between about 5 and about 30 unique SNPs. More preferably, the total number of SNPs in a chromosome-specific panel is about 20 SNPs. In some embodiments, the total number of SNPs in a chromosome-specific panel is, is about, is at least, is at least about, is not more than, is not more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 SNPs, or a range defined by any two of the preceding values.

SNP panels may contain one or more chromosome-specific panels. A chromosome-specific SNP panel can comprise SNPs located on autosomal chromosomes, preferably SNPs located on chromosomes that are susceptible to aneuploidy in clinical relevant syndromes. More preferably, chromosome-specific SNP panels comprise SNPs located on Chromosome 13, 18, 21, X, and Y. A chromosome-specific panel can also be a control chromosome-specific panel. Preferably, a control chromosome-specific panel comprises SNPs located on a chromosome that is not susceptible to aneuploidy or where the aneuploidy is incompatible with viability, which is typically the larger chromosomes that are designated by lower indices (e.g., chromosome 1, 2, or 3). Most preferably, a control chromosome-specific panel comprises SNPs located on Chromosome 1, 2, or 3. In addition, a chromosome-specific SNP panel can also comprise SNPs located on sex-specific chromosomes. In some embodiments, a panel is not specific for a particular chromosome. In some embodiments, the control is not a chromosome-specific SNP panel. For example, primers can be used to amplify a chromosome-specific region which will serve as a control.

In some embodiments, a SNP panel comprises more than one chromosome-specific panel, where the chromosome-specific panels are for SNPs on Chromosome 1, 2, 3, 4, 5, 6, 7, 8, 9, 10, 11, 12, 13, 14, 15, 16, 17, 18, 19, 20, 21, 22, X, or Y. In some embodiments, the SNP panel comprises 1, 2, 3, 4, 5, 6, 7, 8, 9, 10, 11, 12, 13, 14, 15, 16, 17, 18, 19, 20, 21, 22, or 23 chromosome-specific panels, or a range defined by any two of the preceding values. In some embodiments, the total number of SNPs in the panel is N*the number of SNPs on a chromosome-specific panel, where N is the number of chromosome-specific panels in the SNP panel.

SNPs with one allele favored in homozygosity across major known haplotypes can also be selected. In some embodiments, SNPs with a homozygous maternal genotype (pp or qq) at less than 0.25 and an opposite homozygous genotype at more than 0.25 are selected.

In some embodiments, SNPs have a frequency in the range of about 30% to about 50% for the minor allele as measured across all major population groups. In a preferred embodiment, SNPs have a frequency in the range of about 49% to about 50% for the minor allele. In some embodiments, SNPs have a frequency that is, is about, is at least, is at least about, is not more than, is not more than about 30%, 31%, 32%, 33%, 34%, 35%, 36%, 37%, 38%, 39%, 40%, 41%, 42%, 43%, 44%, 45%, 46%, 47%, 48%, 49%, or 50% for the minor allele, or a range defined by any two of the preceding values.

The terms homozygous and heterozygous alleles as used in at least one of the documents cited herein are sometimes referred to herein as heterozygous or homozygous locus or loci, or heterozygous or homozygous genotypes. Further, the term preamplification as used in at least one of the documents cited herein is referred to herein as subsample amplification.

### Invasive Diagnostic Tests

Invasive procedures currently available to diagnose genetic conditions in a fetus include amniocentesis, chorionic villus sampling, and percutaneous umbilical blood sampling. Each test carries an increased risk of miscarriage. Subjects that receive a positive report following a reflex protocol provided herein may further undergo invasive diagnostic testing.

### Amniocentesis

This is a highly invasive procedure in which a needle is passed through the mother's lower abdomen into the amniotic cavity inside the uterus. This procedure can be performed at about 14 weeks gestation. For prenatal diagnosis, most amniocenteses are performed between 14 and 20 weeks gestation. Large chromosomal abnormalities, such as extra or missing chromosomes or chromosome fragments, can be detected by karyotyping, which involves the identification and analysis of all 46 chromosomes from a cell and arranges them in their matched pairs, based on subtle differences in size and structure. In this systematic display, abnormalities in chromosome number and structure are apparent. This procedure typically takes 7-10 days for completion. While amniocentesis can be used to provide direct genetic information, risks are associated with the procedure including miscarriage and maternal Rh sensitization. Amniocentesis carries a one in 200 risk of miscarriage.

### Chorionic Villus Sampling (CVS)

In this invasive procedure, a catheter is passed via the vagina through the cervix and into the uterus to the developing placenta with ultrasound guidance. The introduction of the catheter allows cells from the placental chorionic villi to be obtained and analyzed by a variety of techniques, including chromosome analysis to determine the karyotype of the fetus. The cells can also be cultured for biochemical or molecular biologic analysis. Typically, CVS is performed between 9.5 and 12.5 weeks gestation. CVS carries a one in 100 risk of miscarriage. In some instances, CVS can be associated with limb defects in the fetus. Also, the possibility of maternal Rh sensitization is present. Furthermore, there is also the possibility that maternal blood cells in the developing placenta will be sampled instead of fetal cells and confound chromosome analysis.

### Percutaneous Umbilical Blood Sampling (PUBS)

In this invasive procedure, blood is taken from a vein in the umbilical cord and examined for chromosomal defects (e.g., Down syndrome) and blood disorders (e.g., fetal hemolytic disease). Physicians generally perform this test after 18 weeks of gestation. PUBS carries a 1 to 2 in 100 risk of miscarriage.

### Point of Care System/Point of Care Detector

As one of skill in the art will recognize, the methods described herein can also utilize a point of care system. Further, as one of skill in the art will recognize, the methods described herein can also utilize a point of care detector.

### Computing Systems

The methods described herein are operational with numerous general purpose or special purpose computing system environments or configurations. Examples of well known computing systems, environments, and/or configurations that may be suitable for use with the technology disclosed herein include, but are not limited to, personal computers, server computers, hand-held or laptop devices, multiprocessor systems, microprocessor-based systems, programmable consumer electronics, network PCs, minicomputers, mainframe computers, distributed computing environments that include any of the above systems or devices, and the like.

As used herein, “instructions” refer to computer-implemented steps for processing information in the system. Instructions can be implemented in software, firmware or hardware and include any type of programmed step undertaken by components of the system.

The system can be comprised of various modules/components as discussed in detail. As can be appreciated by one of ordinary skill in the art, each of the modules comprises various sub-routines, procedures, definitional statements and macros. Each of the modules are typically separately compiled and linked into a single executable program. Therefore, the description of each of the modules is used for convenience to describe the functionality of the preferred system. Thus, the processes that are undergone by each of the modules may be arbitrarily redistributed to one of the other modules, combined together in a single module, or made available in, for example, a shareable dynamic link library.

Those of skill will further appreciate that the various logical blocks, modules, circuits, and algorithm steps used in connection with the embodiments disclosed herein may be implemented as electronic hardware, computer software, or combinations of both. To clearly illustrate this interchangeability of hardware and software, various illustrative components, blocks, modules, circuits, and steps may described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In some embodiments, the functions may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Thus, in some embodiments computer readable medium may comprise non-transitory computer readable medium (e.g., tangible media). In addition, in some embodiments, computer readable medium may comprise transitory computer readable medium (e.g., a signal). Combinations of the above should also be included within the scope of computer-readable media.

It will be understood by those of skill in the art that numerous and various modifications can be made without departing from the spirit of the present invention. All the references referred to herein are incorporated by reference in their entirety and for the subject matter discussed. The following examples are included for illustrative purposes only and are not intended to limit the scope of the invention.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Integrated Reflex Protocol Improves Performance of Screening Tests for Fetal Genetic Conditions

To examine the effect of methods described herein on performance of screening tests for fetal genetic conditions, the expected trisomy 21 (Down's syndrome) integrated performance characteristics using an exemplary reflex protocol were estimated.

Estimated integrated performance characteristics included detection rate/sensitivity, true positive rates, false positive rates, true negative rates, false negative rates, and negative predictive values.

Table 1 shows the performance of a screening test currently performed in the first trimester of pregnancy. This first trimester screen combines the results from nuchal translucency, Fβ-hCG, and PAPP-A levels with maternal age risk factors and determines an overall risk factor for chromosomal abnormalities. Table 2 shows the estimated performance of an exemplary integrated screening test using a reflex protocol. Test 1 in this embodiment is an assay that measures the levels of free beta human chorionic gonadotropin in maternal blood. The threshold of the ROC curve for this test has been adjusted to provide 95% sensitivity and 10% specificity. Test 2 is an assay that detects trisomy 21 in cell-free fetal DNA present in maternal blood. Test 3 is an assay that detects trisomy 21 in DNA from fetal cells isolated from maternal blood.

TABLE 1

| | First Trimester Screen |
|---|---|
| Test Population | 1,000,000 |
| Incidence of True | 4,000 |
| Positive | |
| Portion of Biological | plasma/serum |
| Sample | |
| Analyte | Fβ-hCG/PAPP-A |
| Measured/Detected | |
| Detection Rate | 91% |
| (Sensitivity) | |
| False Positive Rate | 5% |
| (Specificity) | |
| Test Negative Predictive | 99.96% |
| Value | |
| True Positives | 3,640 |
| False Positives | 49,818 |
| True Negatives | 946,542 |
| False Negatives | 360 |

As shown in Table 2 below, the methods described herein are effective for improving the performance of screening tests for trisomy 21 (Down's syndrome). Compared to the single screen in Table 1, the performance characteristics of integrated screening using a reflex protocol provided herein are superior. For example, the false positive rate decreases from 10% for a single test screen to 0.004% for the integrated reflex screen. These results indicated that methods and systems provided herein would be effective for improving performance of screening tests for fetal genetic conditions. These results suggested that the methods and systems provided herein are useful for assessing a subject's risk of having a fetus with a genetic condition. The results further suggested that methods and systems provided herein are useful for detecting a genetic condition in the fetus of a subject.

TABLE 2

| | Test 1 | Test 2 | Test 3 |
|---|---|---|---|
| Test Population | 1,000,000 | 103,420 | 5,718 |
| Incidence of True Positive | 4,000 | 3,800 | 3,724 |
| Portion of Biological | plasma/serum | plasma | cellular |
| Sample | | | |
| Analyte | Fβ-hCG | cell-free DNA | DNA from |
| Measured/Detected | | | fetal cells |
| Detection Rate | 95% | 98% | 98% |
| (Sensitivity) | | | |
| False Positive Rate | 10% | 2% | 2% |
| (Specificity) | | | |
| Test Negative Predictive | 99.98% | 99.92% | 96.40% |
| Value | | | |
| Integrated Detection Rate | 95% | 93% | 91% |
| (Sensitivity) | | | |
| Integrated True Positives | 3,800 | 3,724 | 3,650 |
| Integrated False Positives | 99,620 | 1,994 | 41 |
| Integrated True Negatives | 896,580 | 994,282 | 996,309 |
| Integrated False Negatives | 200 | 276 | 350 |
| Integrated False Positive | 10% | 0.2% | 0.004% |
| Rate (Specificity) | | | |
| Integrated Negative | 99.98% | 99.97% | 99.96% |
| Predictive Value | | | |

### Example 2

### Testing Genetic Variations in a Cellular Portion of a Sample

10 nL of a mixture of fetal and maternal cells that has been enriched for fetal cells is distributed into isolated reaction chambers that each contain 90 nL of appropriate buffers and reagents for cell lysis and WGA, such that each chamber contains an average 1 or 0 cells. Cell lysis and a whole genome amplification reaction (WGA) are performed in each chamber. Following WGA, a portion of each amplified sample is transferred to a second reaction chamber containing primers and probes designed to identify a fetal allele as described herein. Samples identified as containing a fetal allele are then subjected to a second WGA and optionally pooled prior to performing array comparative genomic hybridization (aCGH) to identify a genetic variation as described herein.

### Example 3

### Prenatal Screen

Blood samples are collected from pregnant female patients. Plasma or serum is extracted from a portion of each blood sample and divided into plasma or serum sub samples.

PAPP-A and hCG measurements (a “first test”) are taken using a first serum subsample from each patient. Patients identified as having a negative result for the first test are not further tested. A screen for a genetic variation (a “second test”) is then performed in a plasma subsample from each patient identified as having a positive result for the first test. Only patients having a positive result for the second test are advised to consider amniocentesis.

### Example 4

### Prenatal Screen

Blood samples are collected from pregnant female patients. Plasma is extracted from a portion of each blood sample.

A screen for a genetic variation (a “first test”) is performed in a plasma sample from each patient. A screen for a genetic variation (a “second test”) is then performed in a cellular portion of the blood sample from each patient identified as having a positive result for the first test, while patients identified as having a negative result in the first test are not further tested. Only patients having a positive result for the second test are advised to consider amniocentesis.

### Example 5

### Prenatal Screen

Blood samples are collected from pregnant female patients. Plasma is extracted from a portion of each blood sample.

A screen for a genetic variation (a “first test”) is performed in a plasma sample from each patient. A diagnostic test for a genetic condition is then performed in a cellular portion of the blood sample from each patient identified as having a positive result for the first test, while patients identified as having a negative result in the first test are not further tested. Pregnant women having a positive result for the diagnostic test are determined to be carrying a fetus with the genetic condition.

1. A prenatal screening method providing improved accuracy of information for a patient regarding her fetus, comprising:
selecting a plurality of different prenatal tests comprising at least a first prenatal test and a second prenatal test for a prenatal screen of a patient's fetus;
setting a minimum detection rate and a maximum false positive rate for the prenatal screen comprising the plurality of prenatal tests;
adjusting the threshold required for a positive test result for the first prenatal test such that the false positive rate for the first prenatal test exceeds the maximum false positive rate for the prenatal screen;
adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen;
obtaining a biological sample from a patient identified as the source of a test result that meets the adjusted threshold required for a positive test result for the first prenatal test;
subjecting the biological sample from the patient to the second prenatal test to determine a level of a biological marker in the biological sample, where the level of the biological marker constitutes a second prenatal test result;
determining whether the second prenatal test result meets the adjusted threshold required for a positive test result for the second prenatal test;
sequentially subjecting a biological sample from the patient to testing in any remaining plurality of prenatal tests if the test result for the biological sample meets the adjusted threshold required for a positive test result for the second and any subsequent prenatal test; and
identifying the patient as having a positive prenatal screen result if the test results for each of the plurality of prenatal tests to which a biological sample is subjected meets the adjusted threshold required for a positive test result for each of the plurality of prenatal tests,
wherein the false positive rate for the prenatal screen at a given detection rate is less than the false positive rate for any of the plurality of prenatal tests alone, thereby improving the accuracy of information from the prenatal screen of the patient's fetus.

2. The method of claim 1, wherein the adjusted threshold required for a positive test result for the second prenatal test is selected such that the integrated detection rate for the first and second prenatal tests is above the minimum detection rate for the prenatal screen, and such that the integrated false positive rate for the first and second prenatal tests is below the maximum false positive rate for the prenatal screen.

3. The method of claim 1, wherein a third prenatal test is performed following the second prenatal test when the result of the second prenatal test meets the adjusted threshold required for a positive test result for the second prenatal test.

4. The method of claim 3, wherein the third prenatal test is the final prenatal test in the prenatal screen.

5. The method of claim 1, wherein a subsequent prenatal test is not performed in the prenatal screen when a biological sample fails to meet the adjusted threshold required for a positive test result in a prenatal test of the prenatal screen.

6. A method of increasing the cost effectiveness of a prenatal screen, comprising:
selecting a plurality of different prenatal tests comprising at least a first prenatal test and a second prenatal test for a prenatal screen;
setting a minimum detection rate and a maximum false positive rate for the prenatal screen comprising the plurality of prenatal tests;
adjusting the threshold required for a positive test result for the first prenatal test such that the false positive rate for the first prenatal test exceeds the maximum false positive rate for the prenatal screen;
adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen;
generating a first prenatal test result for biological samples from a plurality of patients;
identifying a subset of the biological samples that meets the adjusted threshold required for a positive test result for the first prenatal test and a subset of the biological samples that does not meet the adjusted threshold required for a positive test result for the first prenatal test;
generating a second prenatal test result for the subset of samples that meets the adjusted threshold required for a positive test result for the first prenatal test;
determining whether the second prenatal test result meets the adjusted threshold required for a positive test result for the second prenatal test;
sequentially subjecting samples from the subset of samples that meets the adjusted threshold required for a positive test result for the second prenatal test to testing in any remaining plurality of prenatal tests if the biological samples meet the adjusted threshold required for a positive test result for any subsequent prenatal test; and
identifying patients as having a positive prenatal screen result if the test result for the final prenatal test to which their biological sample is subjected meets the adjusted threshold required for a positive test result,
wherein subsequent prenatal test results are not generated for patients that fail to meet the adjusted threshold required for a positive test result for a prenatal test, thereby reducing the cost of identifying positive prenatal screen results compared to simultaneously generating test results for all of the prenatal tests in all of the patients.

7. (canceled)

8. (canceled)

9. (canceled)

10. A method of reducing the number of unnecessary prenatal screening tests in a patient population, comprising:
selecting a plurality of different prenatal tests comprising at least a first prenatal test and a second prenatal test for a prenatal screen;
setting a minimum detection rate and a maximum false positive rate for the prenatal screen comprising the plurality of prenatal tests;
adjusting the threshold required for a positive test result for the first prenatal test such that the false positive rate for the first prenatal test exceeds the maximum false positive rate for the prenatal screen;
adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen;
generating a first prenatal test result for biological samples from a plurality of patients;
identifying a subset of biological samples that meets the adjusted threshold required for a positive test result for the first prenatal test and a subset of samples that do not meet the adjusted threshold required for a positive test result for the first prenatal test;
generating a second prenatal test result for the subset of samples that meet the adjusted threshold required for a positive test result for the first prenatal test;
determining whether the second prenatal test result meets the adjusted threshold required for a positive test result for the second prenatal test;
sequentially subjecting samples from the subset of samples that meets the adjusted threshold required for a positive test result for the second prenatal test to testing in any remaining plurality of prenatal tests if the biological samples meet the adjusted threshold required for a positive test result for any subsequent prenatal test; and
identifying patients as having a positive prenatal screen result if the test result for the final prenatal test to which their biological sample is subjected meets the adjusted threshold required for a positive test result,
wherein subsequent prenatal test results are not generated for patients that fail to meet the adjusted threshold required for a positive test result for a prenatal test, thereby reducing the number of prenatal tests that must be generated to identify a positive prenatal screen result compared to simultaneously generating test results for all of the prenatal tests in all of the patients.

11. (canceled)

12. A method of decreasing the risk of iatrogenic injury to a normal fetus, comprising:
selecting a plurality of different prenatal tests comprising at least a first prenatal test and a second prenatal test for a prenatal screen;
setting a minimum detection rate and a maximum false positive rate for the prenatal screen comprising the plurality of prenatal tests;
adjusting the threshold required for a positive test result for the first prenatal test such that the false positive rate for the first prenatal test exceeds the maximum false positive rate for the prenatal screen;
adjusting the threshold required for a positive test result for the second and any remaining plurality of prenatal tests such that the integrated detection rate for the plurality of prenatal tests is above the minimum detection rate for the prenatal screen, and such that the integrated false positive rate for the plurality of prenatal tests is below the maximum false positive rate for the prenatal screen;
obtaining a biological sample from a patient identified as the source of a test result that meets the adjusted threshold required for a positive test result for the first prenatal test;
generating a second prenatal test result for the biological sample;
determining whether the second prenatal test result meets the adjusted threshold required for a positive test result for the second prenatal test;
sequentially subjecting a biological sample from the patient to any remaining plurality of prenatal tests if the biological sample meets the adjusted threshold required for a positive test result for the second and any subsequent prenatal test; and
identifying the patient as having a negative prenatal screen result if a test result of the biological sample fails to meet the adjusted threshold required for a positive test result for the second or any subsequent prenatal test; and
identifying the patient as having a positive prenatal screen result if the test results for each of the plurality of prenatal tests to which a biological sample is subjected meets the adjusted threshold required for a positive test result for each of the plurality of prenatal tests,
wherein the false positive rate for the prenatal screen at a given detection rate is less than the false positive rate for any of the plurality of tests alone, thereby improving the accuracy of information from the prenatal screen, and thereby reducing the number of women pregnant with a normal fetus advised to undergo an invasive prenatal procedure, decreasing the risk of iatrogenic injury to the fetus resulting from the invasive prenatal procedure.

13. (canceled)

14. (canceled)

15. A method of screening for a fetal condition of interest, comprising:
(a) obtaining a biological sample;
(b) performing a first prenatal test on the biological sample;
(c) detecting a positive or negative result for the first prenatal test;
(d) reporting a negative test result if the biological sample generates a negative result for the first prenatal test;
(e) performing a second prenatal test if the biological sample generates a positive result for the first prenatal test;
(f) detecting a positive, negative, or inconclusive result for the second prenatal test;
(g) reporting a negative test result if the biological sample generates a negative result for the second prenatal test;
(h) performing a third prenatal test if the biological sample generates a positive or inconclusive result for the second prenatal test;
(i) reporting a negative test result if the biological sample generates a negative result for the third prenatal test;
(j) reporting a positive test result if the biological sample generates a positive result for the third prenatal test;
(k) optionally redrawing the biological sample if the biological sample generates an inconclusive result for the third prenatal test; and
(l) optionally repeating steps (b) through (l) for the redrawn biological sample.

16. The method of claim 1, wherein the biological sample is plasma, serum, or whole blood.

17. (canceled)

18. (canceled)

19. The method of claim 1, wherein the first prenatal test or second prenatal test is a test for at least one biochemical marker, and wherein the biochemical marker is selected from the group consisting of pregnancy-associated plasma protein A (PAPP-A), free beta human chorionic gonadotropin (β-hCG), alpha-fetoprotein (AFP), human chorionic gonadotropin (hCG), unconjugated estriol (UE3), and dimeric inhibin A (DIA).

20. The method of claim 1, wherein the first prenatal test or second prenatal test is a test for a fetal genetic variation in a cellular portion of the biological sample.

21. The method of claim 1, wherein the first prenatal test or second prenatal test is a test for a fetal genetic variation in a cell-free nucleic acid portion of the biological sample.

22. (canceled)

23. (canceled)

24. The method of claim 3, wherein the third prenatal test is a test for a fetal genetic variation in a cellular portion of the biological sample.

25. The method of claim 1, wherein the first prenatal test comprises measuring the concentration level of at least two, three, or four markers.

26. The method of claim 25, wherein the second prenatal test comprises measuring the concentration level of at least two, three, or four markers.

27. (canceled)

28. The method of claim 1, wherein the prenatal screening test is performed to detect a genetic variation.

29-44. (canceled)

45. The method of claim 1, wherein the prenatal screen is diagnostic.
